(19) United States
(12) Patent Application Publication
(10) Pub. No.: US 2012/0117686 A1
(43) Pub. Date: May 10, 2012

(54) STRESS-TOLERANT PLANTS EXPRESSING MANNOSYLGLYCERATE-PRODUCING ENZYMES

(75) Inventors: Henrik Vibe Scheller, Millbrae, CA (US); Jesper Harholt, Frederiksberg (DK); Peter Ulvskov, Frederiksberg (DK)

(73) Assignees: THE REGENTSOF THE UNIVERSITY OF CALIFORNIA, Oakland, CA (US); AARHUS UNIVERSITY, Aarhus (DK); UNIVERSITY OF COPENHAGEN, Copenhagen (DK)

(21) Appl. No.: 13/318,661

(22) PCT Filed: May 4, 2010
(86) PCT No.: PCT/US10/33581

§ 371 Date: Jan. 20, 2012

Related U.S. Application Data
(60) Provisional application No. 61/175,404, filed on May 4, 2009.

(51) Int. Cl. A01H 1/06; A01H 5/00

(52) U.S. Cl. 800/278; 800/298; 800/320; 800/320.1; 800/320.3; 800/320.2; 800/312; 800/314; 800/317.3; 800/317.2; 800/306; 800/322

(57) ABSTRACT

The present invention provides compositions and methods for increasing the level of mannosylglycerate in plants. Plants with increased levels of mannosylglycerate exhibit enhanced tolerance to stress, e.g., drought.

### CROSS-REFERENCE TO RELATED APPLICATIONS

This application claims benefit of U.S. provisional application No. 61/175,404, filed May 4, 2009, which application is herein incorporated by referenced.

### STATEMENT AS TO RIGHTS TO INVENTIONS MADE UNDER FEDERALLY SPONSORED RESEARCH AND DEVELOPMENT

The invention described and claimed herein was made using funds supplied by the U.S. Department of Energy under Contract No. DE-AC02-05CH11231. The government has certain rights in this invention.

### BACKGROUND OF THE INVENTION

Mannosylglycerate (MG) is a compound found in several thermophilic bacteria and archaea and is know to be an efficient thermoprotectant. MG stabilizes proteins and prevents their denaturation and aggregation under stressful conditions, such as heat. MG in bacteria is synthesized by two different routes. The most common route is by mannosyl phosphoglycerate synthase, a glycosyltransferase in family GT55, coupled with a phosphatase. The less common route is a one-step reaction catalyzed by mannosylglycerate synthase, a glycosyltransferase in family GT78. GT55 enzymes are found in archaebacteria, bacteria, and several fungi, but there have been no reports that the enzymes are present in plants or alga. GT78 proteins have been described in bacteria. Recent sequencing of the Physcomitrella (a moss) and Selaginella (a spikemoss) genomes has identified sequences encoding GT78 proteins with high similarity to the known MG synthases. A sequence with similarity to MG synthase gene has also been found in the red alga Griffithsia japonica. GT78 proteins have not been identified in higher plant species, including Arabidopsis and rice. The genus Selaginella contains some of the most drought tolerant plants that are known, including resurrection plants such as S. lepidophylla, which can survive complete desiccation. Physcomitrella is also known to survive severe desiccation. The tolerance of S. lepidophylla has been ascribed to high levels of trehalose in this species, however. There is no description that MG contributes to drought tolerance.

Abiotic stresses seriously impact plant production and limit the areas suitable for plant growth. It is widely recognized that the development of bioenergy crops must not negatively impact food production due to the fact that food production will increase substantially in the future. To meet these demands, it will be necessary to improve plant production in marginal lands that are not currently productive. Development of plants that more efficiently cope with abiotic stresses is an essential component of the ability to grow crops on marginal lands. In addition, water is a limited resource for agriculture on prime agricultural land in large parts of the US. To avoid crop damage and yield losses when insufficient water is available for irrigation, it is crucial to have crop varieties that can sustain temporary drought. Plants respond to drought and heat stress in a variety of ways. A universal response to drought stress in many different organisms involves accumulation of trehalose, which is non-reducing α-glucosyl-α-glucoside. Some drought tolerant plants have the ability to accumulate trehalose, and crops have been made more drought resistant by increasing their trehalose accumulation (Garg et al. 2002, Penna 2003, Almeida et al. 2007). Trehalose can accumulate to high concentrations in the cell and thereby lower the water potential and prevent water loss, while at the same time being compatible with cellular functions.

MG is a compound that is known to be a thermoprotectant, as explained above (Borges et al. 2002). MG stabilizes proteins and prevents their denaturation and aggregation under stressful conditions, especially heat, but also under other stress conditions such as drought, high salt concentration and freezing. For example, the protection by MG, at 0.5 M concentration, against heat inactivation of the model enzyme lactate dehydrogenase (LDH) was compared to that exerted by other compatible solutes, namely, trehalose, ectoine, hydroxyectoine, di-myo-inositol phosphate, diglycerol phosphate, and mannosylglyceramide (Borges et al. 2002). MG was the best stabilizer of the enzyme and accompanied by a higher efficiency in preventing LDH aggregation induced by heat stress. Moreover, MG induced an increase of 4.5° C. in the melting temperature of LDH, whereas the same molar concentration of trehalose caused an increase of only 2.2° C. However, there has been no description of MG as a stabilizer in vivo.

### BRIEF SUMMARY OF THE INVENTION

The invention is based, in part, on the discovery that a seed-producing plant can be genetically modified to produce MG mannosylglycerate (MG) by introducing a mannosylglycerate synthase gene into a plant, which thereby confers stress tolerance, e.g., increased tolerance to drought, on the engineered plant.

As explained above, trehalose is a solute that has been identified as a compatible solute that can confer tolerance to abiotic stress. Solutes need to accumulate to appreciable concentrations in the cell in order to raise the osmotic pressure sufficiently to be of protective value for a water-stressed plant. The solute in question thus must not interfere with cellular function in a detrimental way, including at high concentrations. Thus, this invention, is also based, in part, on the discovery that mannosylglycerate (MG) plays a role as a compatible solute in primitive plants. The invention is further based, in part, on the surprising finding that accumulation of MG in seed plants occurs without compromising viability or agronomic performance, including at concentrations that increase the tolerance of the plant to temporary drought and other water-balance stresses (e.g., heat, freezing, high salt).

The MG synthase protein is directed to different subcellular compartments by generating use of chimeric gene sequences to target the compartment. Typically, a MG synthase from Selaginella or Physcomitrella is engineered into the plant, although in additional embodiments, a bacterial MG synthase gene or MG synthase gene from red algae is employed.

In some embodiments, a plant with improved stress tolerance can be engineered using a gene encoding mannosyl-3-phosphoglycerate synthase (also referred to herein as a GT55 gene or a GT55 family member), which catalyzes the conversion of GDP mannose and D-3-phosphoglycerate into a phosphorylated intermediate, and a gene encoding a phosphatase that is active on mannosyl-phophoglycerate to convert the intermediate to mannosylglycerate.

In typical embodiments, the gene encoding the MG synthesizing enzyme(s) that is introduced into a plant is codon-optimized for expression in the plants.

In some embodiment the plant that is engineered is a crop plant. For example, suitable plants include corn, switchgrass, sorghum, miscanthus, sugarcane, poplar, pine, wheat, rice, soy, cotton, barley, turf grass, tobacco, potato, bamboo, rape, sugar beet, sunflower, willow, and eucalyptus. In further embodiments, the plant is switchgrass (Panicum virgatum), giant reed (Arundo donax), reed canarygrass (Phalaris arundinacea), Miscanthus x giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, or Kentucky bluegrass among others.

In some embodiments the plant is selected among species and cultivars that already accumulate a compatible solute, e.g., trehalose, when exposed to salt-, heat-, cold- or drought-stress.

In one embodiment, a plant that is genetically modified to produce MG plant is also engineered to reduce, or prevent, production of an unwanted solute, e.g., glycine betaine. Accordingly, beet, spinach, wheat, barley, corn, sugarcane, sunflower and cotton transgenic plants that produce MG may also have been genetically modified to reduce glycine betaine levels.

In other embodiments, a plant such as the grasspea Lathyrus sativus can be genetically modified to produce MG. In some embodiments, the grasspea plant may be a cultivar that lacks the non-protein amino acid, 2-amino-2-carboxyethyloxamid acid. In other embodiments, the grass pea may be genetically modified by using recombinant technology to prevent or reduce 2-amino-2-carboxyethyloxamid acid production.

In another embodiment where the transgenic plant is engineered to accumulate MG, the accumulation of mannosylglycerate is in addition to, i.e., “stacked on top of”, accumulation of an endogenous compatible solute where this latter solute is not undesired. For example, the disaccharide trehalose is a compatible solute commonly used by seed plants that typically has no unwanted side effects. Accordingly, in one embodiment, MG accumulation is in addition to trehalose accumulation. In some embodiments, trehalose and MG may have a synergistic effect on carbohydrate metabolism and stress tolerance.

In one aspect, the invention thus provides a method of increasing stress tolerance in a plant, the method comprising expressing a heterologous MG synthase gene in the plant, thereby increasing the tolerance of the plant to stress. In some embodiments, the MG synthase gene encodes a protein having MG synthase activity and at least 60%, 65%, 70%, 75%, 80%, 85%, 90%, 95%, or greater, identity to the amino acid sequence of SEQ ID NO:1, SEQ ID NO:2, SEQ ID NO:3, or SEQ ID NO:4. In some embodiments, the MG synthase gene encodes a protein having MG synthase activity and at least 60%, 65%, 70%, 75%, 80%, 85%, 90%, 95%, or greater, identity to the amino acid sequence of SEQ ID NO:1, SEQ ID NO:2, SEQ ID NO:3, or SEQ ID NO:4; and further, comprises the sequence RHYFPRSSTDAMITWF or VRHYFPRxSTDAMITWF; or the sequence EVYIPEGKVHALYSGLRDLRTMLVECFSAMQSLK or ExYIxEGKxHxLYxGLxDLRTMLVECFxAxQSL. In some embodiments, the MG synthase gene encodes a protein having MG synthase activity and at least 60%, 65%, 70%, 75%, 80%, 85%, 90%, 95%, or greater, identity to the amino acid sequence of SEQ ID NO:1, SEQ ID NO:2, SEQ ID NO:3, or SEQ ID NO:4; and further, comprises the sequence RHYFPRSSTDAMITWF or VRHYFPRxSTDAMITWF; and the sequence EVYIPEGKVHALYSGLRDLRTMLVECFSAMQSLK or ExYIxEGKxHxLYxGLxDLRTMLVECFxAxQSL. In some embodiments, the MG synthase gene encodes a protein that has MG synthase activity and comprises the sequence RHYFPRSSTDAMITWF or VRHYFPRxSTDAMITWF; and the sequence EVYIPEGKVHALYSGLRDLRTMLVECFSAMQSLK or ExYIxEGKxHxLYxGLxDLRTMLVECFxAxQSL In some embodiments, the MG synthase gene encodes a protein that has at least 90% identity, or at least 95% identity, to the amino acid sequence SEQ ID NO:1, SEQ ID NO:2, SEQ ID NO:3, or SEQ ID NO:4. In some embodiments, the MG synthase gene encodes a protein comprising the amino acid sequence of SEQ ID NO:1, SEQ ID NO:2, SEQ ID NO:3, or SEQ ID NO:4. In some embodiments, the plant is corn, switchgrass, sorghum, miscanthus, sugarcane, poplar, pine, wheat, rice, soy, cotton, barley, turf grass, tobacco, potato, bamboo, rape, sugar beet, sunflower, willow, or eucalyptus. In some embodiments, the MG synthase gene is operably linked to a drought-inducible promoter.

In a further aspect, the invention provides a plant comprising an expression cassette comprising a heterologous MG synthase nucleic acid sequence that encodes a protein having MG synthase activity and at least 60%, 65%, 70%, 75%, 80%, 85%, or 90%, 95%, or greater, identity to the amino acid sequence of SEQ ID NO:1, SEQ ID NO:2, SEQ ID NO:3, or SEQ ID NO:4. In some embodiments, the MG synthase gene nucleic acid encodes a protein having MG synthase activity and at least 60%, 65%, 70%, 75%, 80%, 85%, 90%, 95%, or greater, identity to the amino acid sequence of SEQ ID NO:1, SEQ ID NO:2, SEQ ID NO:3, or SEQ ID NO:4; and further, comprises the sequence RHYFPRSSTDAMITWF or VRHYFPRxSTDAMITWF; or the sequence EVYIPEGKVHALYSGLRDLRTMLVECFSAMQSLK or ExYIxEGKxHxLYxGLxDLRTMLVECFxAxQSL. In some embodiments, the MG synthase nucleic acid encodes a protein having MG synthase activity and at least 60%, 65%, 70%, 75%, 80%, 85%, 90%, 95%, or greater, identity to the amino acid sequence of SEQ ID NO:1, SEQ ID NO:2, SEQ ID NO:3, or SEQ ID NO:4; and further, comprises the sequence RHYFPRSSTDAMITWF or VRHYFPRxSTDAMITWF; and the sequence EVYIPEGKVHALYSGLRDLRTMLVECFSAMQSLK or ExYIxEGKxHxLYxGLxDLRTMLVECFxAxQSL. In some embodiments, the MG synthase nucleic acid encodes a protein that has at least 90% identity, or at least 95% identity, to the amino acid sequence SEQ ID NO:1, SEQ ID NO:2, SEQ ID NO:3, or SEQ ID NO:4. In some embodiments, the MG synthase nucleic acid encodes a protein comprising the amino acid sequence of SEQ ID NO:1, SEQ ID NO:2, SEQ ID NO:3, or SEQ ID NO:4. In some embodiments, the plant is corn, switchgrass, sorghum, miscanthus, sugarcane, poplar, pine, wheat, rice, soy, cotton, barley, turf grass, tobacco, potato, bamboo, rape, sugar beet, sunflower, willow, or eucalyptus. In some embodiments, the plant comprises an MG synthase gene that is operably linked to a drought-inducible promoter.

In yet another aspect, the invention provides a method of increasing stress tolerance in a plant, the method comprising expressing a heterologous mannosyl-phospho-glycerate synthase gene in the plant, wherein the plant comprises a phosphatase that converts mannosyl-3-phophoglycerate to mannosylglycerate. In some embodiments, the phosphatase is expressed by a heterologous nucleic acid present in the plant. The plant can be, e.g., corn, switchgrass, sorghum, miscanthus, sugarcane, poplar, pine, wheat, rice, soy, cotton, barley, turf grass, tobacco, potato, bamboo, rape, sugar beet, sunflower, willow, or eucalyptus.

The invention further provides a plant comprising a heterologous mannosyl-phospho-glycerate synthase gene and wherein the plant comprises a phosphatase that converts mannosyl-3-phophoglycerate to mannosylglycerate. In some embodiments, the phosphatase is encoded by a heterologous gene. The plant can be, e.g., corn, switchgrass, sorghum, miscanthus, sugarcane, poplar, pine, wheat, rice, soy, cotton, barley, turf grass, tobacco, potato, bamboo, rape, sugar beet, sunflower, willow, or eucalyptus.

### BRIEF DESCRIPTION OF THE DRAWINGS

FIG. 1 provides an alignment of Physcomitrella, Selaginella moellendorffii, Rhodothermus marinus and partial Griffithsia japonica MG synthase protein sequences (SEQ ID NOS:33-34). SEQ ID NO:1 is the Selaginella moellendorffii polypeptide sequence set forth in FIG. 1. SEQ ID NO:2 is the Physcomitrella patens polypeptide sequence set forth in FIG. 1. SEQ ID NO:3 is the Rhodothermus marinus polypeptide sequence set forth in FIG. 1.

FIG. 2 provides data showing expression of mRNA from a MG synthase gene in Arabidopsis. RNA was isolated from Arabidopsis leaves, treated with DNAse, and used to synthesize cDNA using reverse transcriptase. The cDNA was subjected to PCR using the following program: 98° C. for 30 sec initial denaturation, then 98° C. for 10 sec, 55° C. for 30 sec, 70° C. for 60 sec, 40 cycles in total, final extension 70° C. for 10 min using specific primers. The primers used were: Actin-F, CTCCCGCTATGTATGTCGCC (SEQ ID NO:17); Actin R, CAGAATCCAGCACAATACCGGT (SEQ ID NO:18); Ubiqutin-F, GGCCTTGTATAATCCCTGATGAATAAG (SEQ ID NO:19); Ubiqutin-R AAAGAGATAACAGGAACGGAAACATAGT (SEQ ID NO:20); GT78-F CACCTCTCTTGTTTGTTTTCC (SEQ ID NO:21); GT78-R, TCCAGTTGAAGATTCG (SEQ ID NO:22). The PCR products were separated on an agarose gel and stained with Gel-red™. A and B, transgenic Arabidopsis plants transformed with the construct for expression of MG synthase without (A) or with (B) HA tag. C, untransformed control plant. D, positive control (transformation vector). 1, primers for actin; 2, primers for ubiquitin; 3, primers for codon optimized GT78A1; 4, markers.

FIG. 3 provides data showing expression of MG synthase protein. Leaves were harvested 3 days after infiltration with Agrobacterium carrying the pEarlygate201-35S-HA-GT78A1 plasmid. The leaves (50 mg) were ground to a powder in liquid nitrogen and the powder extracted with a buffer of 10 mM Tris-HCl pH 8.0, 150 mM NaCl, 0.1% NP40, 1 mM EDTA. The extracts were recovered by centrifugation and the protein concentration determined using a protein determination kit from Bio-Rad. Samples containing about 20 μg total protein were mixed with SDS sample buffer and subjected to SDS-polyacrylamide gel electrophoresis on 8-16% Tris-glycine Novex Midi gels (Invitrogen). The gel was blotted onto PVDF membrane and the presence of the HA tag was examined using a primary antibody against HA (Sigma-Aldrich) and horse-radish peroxidase coupled pig-anti rabbit IgG secondary antibodies (Sigma-Aldrich) and chemiluminescence detection. M, molecular mass markers; C, control plant infiltrated only with buffer; 1-8, individual plants infiltrated with the pEarlygate201-35S-HA-GT78A1 plasmid.

FIG. 4 shows the results of an analysis of extracts prepared from leaves as described in Example 3. Extracts were analyzed by LS-MS. The upper panel show elution of the MG peak corresponding to elution time 4.73 min and the monoisotopic mass peak determined to 267.07381. The middle panel show analysis of extract from a transgenic arabidopsis plant expressing GT78A1 and MG determined at 267.07082. The bottom panel show analysis of extract from an untransformed arabidopsis plant, no MG could be detected in this sample. The inserts show the mass peaks distribution with the monoisotopic peak and the smaller peaks corresponding to C13 containing MG.

FIG. 5 shows the results of a PCR analysis to confirm the transgenic state of Brachypodium into which a plasmid encoding MG synthase was introduced.

### DETAILED DESCRIPTION OF THE INVENTION

### Definitions

The term “mannosylglycerate synthase gene” in the context of this invention refers to a nucleic acid that encodes a mannosylglycerate (MG) synthase protein, or fragment thereof. Thus, such a gene is often a cDNA sequence that encodes MG synthase. In other embodiments, a MG synthase gene may include sequences such as introns that are not present in a cDNA.

An “MG synthase polypeptide” or “GT78 polypeptide” is an amino acid sequence encoded by a MG synthase nucleic acid. In some embodiments, an MG synthase polypeptide comprises the amino acid sequence of SEQ ID NO:1, SEQ ID NO:2, or SEQ ID NO:3 or is substantially similar to SEQ ID NO:1, SEQ ID NO:2, or SEQ ID NO:3, or a fragment or domain thereof that has MG synthase activity. Thus, a MG synthase polypeptide can, for example: 1) have at least 55% identity, typically at least 60%, 65%, 70%, 75%, 80%, 85%, 90%, 95% or greater identity to SEQ ID NO:1, SEQ ID NO:2, or SEQ ID NO:3, or over a comparison window of at least 100, 200, 250, 300, or 350 or more amino acids of SEQ ID NO:1, SEQ ID NO:2, or SEQ ID NO:3; or 2) comprise at least 100, typically at least 200, 250, 300, or 350, or more contiguous amino acids of SEQ ID NO:1, SEQ ID NO:2, or SEQ ID NO:3; or 3) bind to antibodies raised against an immunogen comprising an amino acid sequence of SEQ ID NO:1, SEQ ID NO:2, or SEQ ID NO:3. A MG synthase polypeptide in the context of this invention is a functional protein that catalyzes the conversion of GDP mannose and D-glycerate to mannosylglycerate. As understood in the art, although the examples of MG synthase amino acid sequences provided herein may show a start “M” as the first amino acid, the start “M” may be removed and not present in the mature protein.

A “MG synthase polypeptide” encoded by a nucleic acid construct of the invention may also be a bifunctional polypeptide that has a mannosyl-3-phosphoglycerate synthase activity and a mannosyl-3-phosphoglycerate phosphatase activity. Thus, in some embodiments, an MG synthase polypeptide expressed in a transgenic plant of the invention comprises the amino acid sequence of SEQ ID NO:4 or is substantially similar to SEQ ID NO:4, or a fragment or domain thereof that has MG synthase activity. Thus, a MG synthase polypeptide can, for example: 1) have at least 55% identity, typically at least 60%, 65%, 70%, 75%, 80%, 85%, 90%, 95% or greater identity to SEQ ID NO:4, or over a comparison window of at least 100, 200, 250, 300, or 350 or more amino acids of SEQ ID NO:4; or 2) comprise at least 100, typically at least 200, 250, 300, or 350, or more contiguous amino acids of SEQ ID NO:4; or 3) bind to antibodies raised against an immunogen comprising an amino acid sequence of SEQ ID NO:4. A MG synthase polypeptide in the context of this invention is a functional protein that catalyzes the conversion of GDP mannose and D-glycerate to mannosylglycerate.

As used herein, a homolog or ortholog of a MG synthase gene is a second gene in the same plant type or in a different plant type that is substantially identical (determined as described below) to a sequence in the first gene.

“GT55” is used interchangeably herein with “mannosyl phospho-glycerate synthase” to refer to fragments, variants, and the like of GT55 family members. A GT55 family member is a glycosyltransferase that catalyzes the synthesis of mannosyl phospho-glycerate. GT55 family members have been identified in bacteria, archaebacteria, and some fungi. In some embodiments, a GT55 protein for use in the invention is a bifunctional mannosyl-phospho-glycerate synthase/mannosyl-phospho-glycerate phosphatase, e.g., from Dehalococcoides ethenogenes (Empadinhas et al., 2004) and thus, the protein also includes the phosphatase activity. Additional exemplary GT55 family members are further described herein below.

The term “mannosyl phosphoglycercate phosphatase” refers to nucleic acids and polypeptides fragments, variants, and the like of phosphatases that are active on mannosyl-phospho-glycerate to convert it to mannosylglycerate.

The term “compatible solute” is used here to refer to major intracellular organic solutes that accumulate in response to osmotic stress, including heat, salt, frost and drought. The compatible solutes, also called osmolytes, include sugars, amino acids and their derivatives, polyols and their derivatives and betaines. Compatible solutes thus refer to low-molecular-weight organic compounds that accumulate, or can be made to accumulate, to high intracellular levels under osmotic stress and that are compatible with the metabolism of the cell. The term covers inter alia betaine, sarcosine, trehalose, mannosylglycerate, hydroxyproline, hydroxylysine and glycosides of the hydroxy-aminoacids, proline and derivatives of inositol.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

The terms “nucleic acid” and “polynucleotide” are used synonymously and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides, that permit correct read through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell. In the context of this invention, the term “mannosylglyercerate synthase coding region” when used with reference to a nucleic acid reference sequence refers to the region of the nucleic acid that encodes the protein.

The term “promoter” or “regulatory element” refers to a region or sequence determinants located upstream or downstream from the start of transcription that direct transcription. As used herein, a promoter includes necessary nucleic acid sequences near the start site of transcription, such as, in the case of a polymerase II type promoter, a TATA element. A promoter also optionally includes distal elements, which can be located as much as several thousand base pairs from the start site of transcription. A “constitutive” promoter is a promoter that is capable of initiating transcription in most environmental and developmental conditions and in nearly all tissue types, whereas a “tissue-specific promoter” initiates transcription only in one or a few particular tissue types. An “inducible” promoter is a promoter that is active under environmental or developmental regulation. The term “operably linked” refers to a functional linkage between a nucleic acid expression control sequence (such as a promoter) and a second nucleic acid sequence, such as a MG synthase, gene, wherein the expression control sequence directs transcription of the nucleic acid corresponding to the second sequence. A “plant promoter” is a promoter capable of initiating transcription in plant cells. Such a promoter is therefore active in a plant cell, but need not originate from that organism. It is understood that limited modifications can be made without destroying the biological function of a regulatory element and that such limited modifications can result in regulatory elements that have substantially equivalent or enhanced function as compared to a wild type regulatory element. These modifications can be deliberate, as through site-directed mutagenesis, or can be accidental such as through mutation in hosts harboring the regulatory element. All such modified nucleotide sequences are included in the definition of a plant regulatory element as long as the ability to confer expression in plant is retained.

“Increased” or “enhanced” activity or expression of a mannosylglycerate-producing enzymes refers to an increase in activity of an enzyme that produces mannosylglyercerate. Examples of such increased activity or expression include the following. Enzyme activity or expression of a gene encoding the enzyme is increased above the level of that in wild-type, non-transgenic control plant (i.e., the quantity of enzyme activity or expression of the gene encoding the enzyme is increased). Enzyme activity or expression of a gene encoding the enzyme is also considered to be “increased” in expression in a cell when it is not normally detected in wild-type, non-transgenic cells. In addition, enzyme activity or expression is also considered to be increased when enzyme activity or expression of a gene encoding the enzyme is present in a cell for a longer period than in a wild-type, non-transgenic controls (i.e., duration of enzyme activity or expression of a gene encoding the enzyme is increased).

“Expression” of a mannosylglycerate-producing enzyme in the context of this invention typically refers to introducing one or more genes that encoding mannosylglycerate-producing enzymes into a higher plant in which it is not normally expressed. Accordingly, an “increase” in mannosylglycerate-producing activity or expression is generally determined relative to wild type cells that have no mannosylglycerate-producing enzyme activity.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

A polynucleotide “exogenous” to an individual plant is a polynucleotide which is introduced into the plant by any means other than by a sexual cross. Examples of means by which this can be accomplished are described below, and include Agrobacterium-mediated transformation, biolistic methods, electroporation, and the like. Such a plant containing the exogenous nucleic acid is referred to here as a T₁ (e.g., in Arabidopsis by vacuum infiltration) for the first generation transformant, T2 for transformants raised from T1 seeds etc.

As used herein, the term “transgenic” describes a non-naturally occurring plant that contains a genome modified by man, wherein the plant includes in its genome an exogenous nucleic acid molecule, which can be derived from the same or a different plant species. The exogenous nucleic acid molecule can be a gene regulatory element such as a promoter, enhancer, or other regulatory element, or can contain a coding sequence, which can be linked to a heterologous gene regulatory element. Transgenic plants that arise from sexual cross or by selling are descendants of such a plant and are also considered “transgenic.”.

An “expression cassette” refers to a nucleic acid construct, which when introduced into a host cell, results in transcription and/or translation of a RNA or polypeptide, respectively.

In the case of expression of transgenes one of skill will recognize that the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “MG synthase polynucleotide sequence” or “MG synthase gene”.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. A “comparison window”, as used herein, includes reference to a segment of any one of the number of contiguous positions, e.g., 20 to 600, usually about 50 to about 200, more usually about 100 to about 150 in which a sequence may be compared to a reference sequence of the same number of contiguous positions after the two sequences are optimally aligned.

The term “substantial identity” in the context of polynucleotide or amino acid sequences means that a polynucleotide or polypeptide comprises a sequence that has at least 50% sequence identity to a reference sequence. Alternatively, percent identity can be any integer from 50% to 100%. Exemplary embodiments include at least: 55%, 57%, 60%, 65%, 70%, 75%, 80%, 85%, 90%, 95%, or 99% identity compared to a reference sequence using the programs described herein; preferably BLAST using standard default parameters, as described below. Accordingly, MG synthase sequences of the invention include nucleic acid sequences that have substantial identity to the MG synthase coding regions of SEQ ID NO:5, SEQ ID NO:6, or SEQ ID NO:7. MG synthase polypeptide sequences of the invention include polypeptide sequences having substantial identity to SEQ ID NO:1, SEQ ID NO:2, SEQ NO:3, or SEQ ID NO:4.

Polypeptides that are “substantially similar” share sequences as noted above except that residue positions that are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 55° C., 60° C., or 65° C. Such washes can be performed for 5, 15, 30, 60, 120, or more minutes.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. For example, a MG synthase polynucleotides, can also be identified by their ability to hybridize under stringency conditions (e.g., Tm ˜40° C.) to nucleic acid probes having the sequence of SEQ ID NO:5, SEQ ID NO:6, or SEQ ID NO:7. Such a MG synthase nucleic acid sequence can have, e.g., about 25-30% base pair mismatches or less relative to the selected nucleic acid probe. SEQ ID NO:5 is an exemplary MG synthase polynucleotide sequence. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

The term “isolated”, when applied to a nucleic acid or protein, denotes that the nucleic acid or protein is essentially free of other cellular components with which it is associated in the natural state. It is preferably in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest.

As used herein, the term “drought-resistance” or “drought-tolerance,” including any of their variations, refers to the ability of a plant to recover from periods of drought stress (i.e., little or no water for a period of days). Typically, the drought stress will be at least 5 days and can be as long as, for example, 18 to 20 days or more (e.g., at least 6, 7, 8, 9, 10, 11, 12, 13, 14, 15, 16, 17, 18, 19, 20 days), depending on, for example, the plant species.

“Heat tolerance”, “cold tolerance” and “salt tolerance” refer to the ability of a plant to recover from periods of heat stress, cold stress, or high salt stress.

Plants That Can Be Engineered in Accordance with the Invention

Various kinds of plants can be engineered to express a mannosylglyercerate-producing enzyme, e.g., a GT78, or a GT55 family member. The plant may be a monocotyledonous plant or a dicotyledonous plant. In certain embodiments of the invention, plants are green field plants.

In some embodiments, plants are grown specifically for “biomass energy”. For example, suitable plants include corn, switchgrass, sorghum, miscanthus, sugarcane, poplar, pine, wheat, rice, soy, cotton, barley, turf grass, tobacco, potato, bamboo, rape, sugar beet, sunflower, willow, and eucalyptus. In further embodiments, the plant is switchgrass (Panicum virgatum), giant reed (Arundo donax), reed canarygrass (Phalaris arundinacea), Miscanthus x giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, or Kentucky bluegrass among others.

### MG Synthase and GT55/Mannosyl Phosphoglyercetase Phosphatase Pathway Nucleic Acid Sequences

The invention employs various routine recombinant nucleic acid techniques. Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel, et al., John Wiley and Sons, New York, 2009).

MG synthase nucleic acid and polypeptide sequences are known in the art. MG synthase genes have been identified in Physcomitrella and Selaginella, as well as Rhodothermus marinus. A sequence with similarity to MG synthase gene has also been found in the red alga Griffithsia japonica.

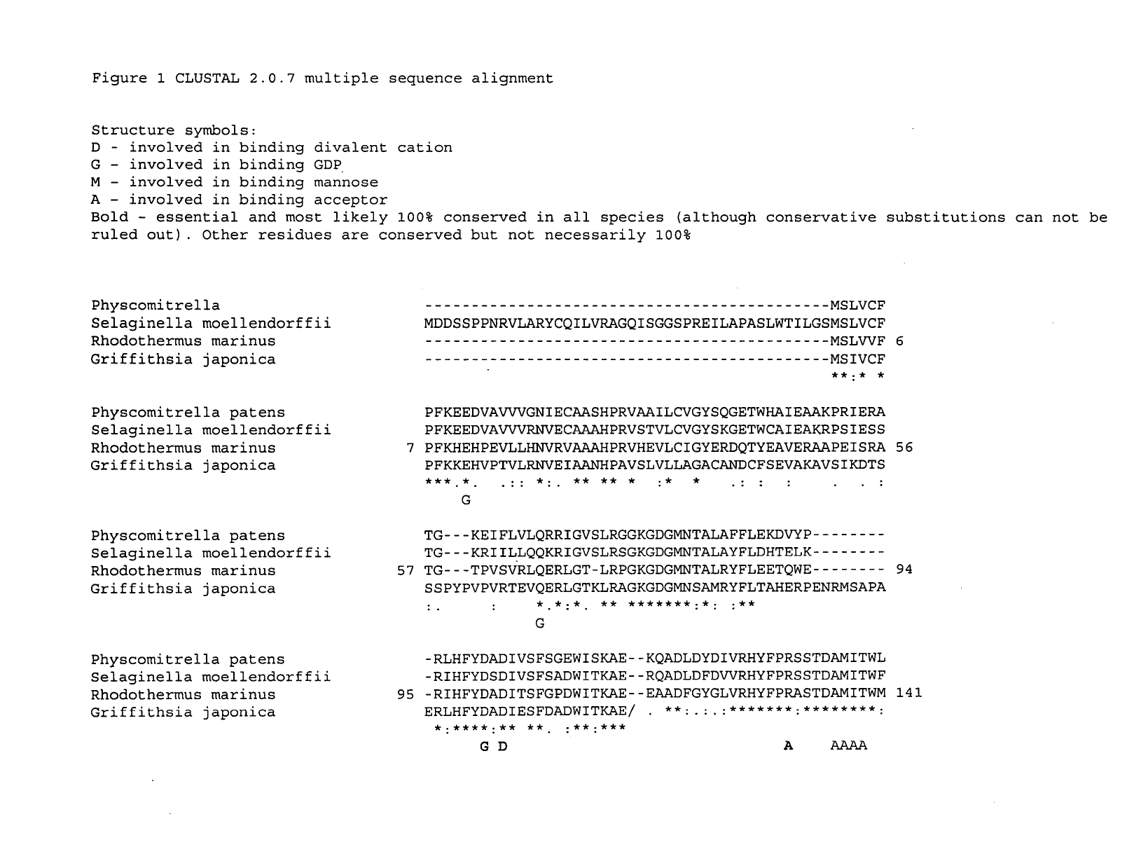

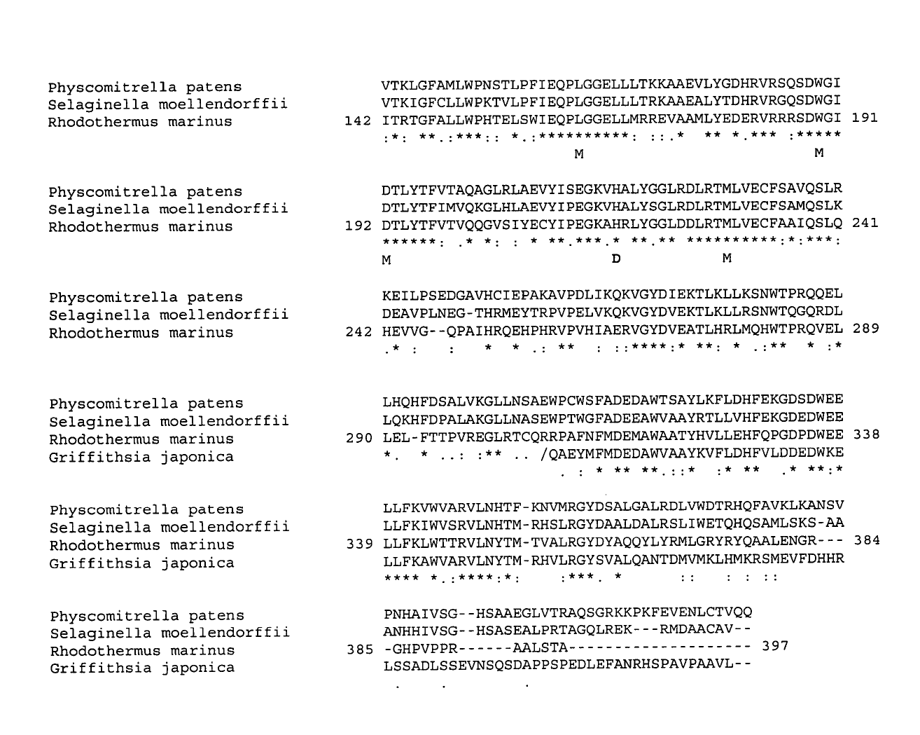

A comparison of Physocmitrella patens, Selaginella moellendorffii, Rhodothermus marins is provided in FIG. 1. As shown in FIG. 1, there are highly conserved regions of the MG polypeptide sequences. For example, the sequences RHYFPRSSTDAMITWF (or VRHYFPRxSTDAMITWF) and EVYIPEGKVHALYSGLRDLRTMLVECFSAMQSLK (or ExYIxEGKxHxLYxGLxDLRTMLVECFxAxQSL) [SEQ ID NOS:23-26, respectively] are highly conserved in the bacterial Rhodothermus marinus sequence and Selaginella and Physcomitrella sequences.

The structure of Rhodthermus marinus had also been determined. In FIG. 1, residues that make contact with the substrates GDP-mannose and glycerate are indicated as noted in the symbol legend in FIG. 1. The protein has two domains. The N-terminal half has all of the substrate binding sites and catalytic residues. The C-terminal half is an interaction domain for making the oligomeric form of the enzyme. Accordingly, a GT78 protein for use in the invention may lack C-terminal residues, or may have mutations in the C-terminal region that do not influence catalytic activity. Other mutations in MG synthase that preserve function include substitutions, typically conservative substitutions, e.g., at positions in the amino acid sequences SEQ NO:1 and SEQ ID NO:2, that are not strictly conserved. Other variants include amino acid substitutions based on the residue at that position in other GT78 family members.

In some embodiments of the invention, a nucleic acid sequence that encodes a Selaginella or Physocomitrella MG synthase protein such as SEQ ID NO:1 or SEQ ID NO:2 is used. In other embodiments, a nucleic acid sequence that encodes a bacterial or algal MG synthase protein such as SEQ ID NO:3 or an MG synthase sequence from Griffithsia japonica is used. The MG synthase polypeptides encoded by the nucleic acids employed in the methods of the invention have the catalytic activity of converting GDP-mannose and D-glycerate into mannosylglycerate. Typically, the level of activity is equivalent to the activity exhibited by a Selaginella or Physocomitrella MG synthase polypeptide (e.g., an MG synthase polypeptide comprising the sequence of SEQ ID NO: 1 or SEQ ID NO:2).

In some embodiments of the invention, a transgenic plant that produced MG is obtained by introducing an expression vector that encodes a MG synthase gene that is a member of the GT55 family. For example, Dehalococcoides ethenogenes has a gene, mgsD, that encodes a bifunctional MG synthase that has two domains, a mannosyl-3-phosphoglycerate synthase domain and a mannosyl-3-phosphoglycerate phosphatase domain. These domains catalyze the consecutive synthesis and dephosphorylation of mannosyl-3-phosphoglycerate to yield MG. Accordingly, in some embodiments, a nucleic acid encoding a bifunctional MG synthase, e.g., the Dehalococcoides ethenogenes MG synthase having a sequence of SEQ ID NO:4 (J. Bacteriol., 186:4075-4084, 2004; see also EP 1526180) is introduced into a plant.

A transgenic plant of the invention may also encode a GT55 family member. Many GT55 family members are known. These include GT55 family members from Thermus and Pyrococcus sp, (e.g., accession numbers AA043097, CAB50138, AAL80715, AAY4481, BAA30023); GT55 family members from Dehalococcoides (accession numbers CAI83370, ABQ17753, AAW3938); GT55 family members from Palaeococcus ferrophilus (accession number AAY44), GT55 family members from various other archaea (accession numbers AAU823, AAU8441, AAU827, CAD42, CAF28, BAE95); GT55 family members from Rodothermus (accession number AAP74552); Staphlothermus (accession number ABN696); Aeropyrum (accession number BAA79872.2); Magnaporthe (accession number XP_{—}362336); Neurospora (accession number XP_{—}325555); and Podospora (accession number CAP65753). Typically, any protein with more than 40% identity, typically more than 45%, 50%, 60%, 70%, 75%, or 80%, or greater, to any of these exemplary protein sequences over a region of 200 residues as defined by a blast alignment using default parameters is a GT55 family member.

Genetic modification of plant to express GT55 is often performed in conjunction with modifying the plant to express a phosphatase that can remove the phosphate from the mannosyl-phosphoglycerate substrate. Examples of such phosphatases include the phosphatase domain of SEQ ID NO:4, as well as many other phosphatases. Other examples of phosphatases include those having accession numbers YP_{—}182074 (Dehalococcoides), ABE51701 (Methanococcoides), AA043098 (Thermus), NP_{—}126909 (Pyrococcus_{—}abyssi), and AAP74553 (Rhodothermus).

Isolation or generation of MG synthase or GT55 and phosphatase polynucleotide sequences can be accomplished by a number of techniques. Cloning and expression of such technique will be addressed in the context of MG synthase genes. However, the same techniques can be used to isolate and express GT55 family members as well as phosphatases that are active on mannosyl-3-phophoglycerate. For instance, oligonucleotide probes based on the sequences disclosed here can be used to identify the desired polynucleotide in a cDNA or genomic DNA library from a desired plant species. Such a cDNA or genomic library can then be screened using a probe based upon the sequence of a cloned MG synthase gene, e.g., SEQ ID NO:5 or SEQ ID NO:6. Probes may be used to hybridize with genomic DNA or cDNA sequences to isolate homologous genes in the same or different plant species.

Alternatively, the nucleic acids of interest can be amplified from nucleic acid samples using amplification techniques. For instance, PCR may be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying a MG synthase gene from plant cells such as moss or spikemoss, can be generated from comparisons of the sequences provided herein. For a general overview of PCR see PCR Protocols: A Guide to Methods and Applications. (Innis, M, Gelfand, D., Sninsky, J. and White, T., eds.), Academic Press, San Diego (1990).

MG synthase nucleic acid sequences for use in the invention includes genes and gene products identified and characterized by techniques such as hybridization and/or sequence analysis using exemplary nucleic acid sequences, e.g., SEQ ID NO:5, SEQ ID NO:6, or SEQ ID NO:7.

### Preparation of Recombinant Vectors

To use isolated sequences in the above techniques, recombinant DNA vectors suitable for transformation of plant cells such as crop plant cells are prepared. Techniques for transformation are well known and described in the technical and scientific literature. For example, a DNA sequence encoding a MG synthase gene (described in further detail below), can be combined with transcriptional and other regulatory sequences which will direct the transcription of the sequence from the gene in the intended cells, e.g., grass or other crop plant cells. In some embodiments, an expression vector that comprises an expression cassette that comprises the MG synthase gene further comprises a promoter operably linked to the MG synthase gene. In other embodiments, a promoter and/or other regulatory elements that direct transcription of the MG synthase gene are endogenous to the plant and an expression cassette comprising the MG synthase gene is introduced, e.g., by homologous recombination, such that the heterologous MG synthase gene is operably linked to an endogenous promoter and is expression driven by the endogenous promoter.

Regulatory sequences include promoters, which may be either constitutive or inducible, or tissue-specific. In some embodiments, a promoter can be used to direct expression of MG synthase nucleic acids under the influence of changing environmental conditions.

### Constitutive Promoters

A promoter, or an active fragment thereof, can be employed which will direct expression of a nucleic acid encoding a fusion protein of the invention, in all transformed cells or tissues, e.g. as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless, Arch. Virol. 142:183-191, 1997); the 1′- or 2′-promoter derived from T-DNA of Agrobacterium tumefaciens (see, e.g., Mengiste supra (1997); O'Grady, Plant Mol. Biol. 29:99-108, 1995); the promoter of the tobacco mosaic virus; the promoter of Figwort mosaic virus (see, e.g., Maiti, Transgenic Res. 6:143-156, 1997); actin promoters, such as the Arabidopsis actin gene promoter (see, e.g., Huang, Plant Mol. Biol. 33:125-139, 1997); alcohol dehydrogenase (Adh) gene promoters (see, e.g., Millar, Plant Mol. Biol. 31:897-904, 1996); ACT11 from Arabidopsis (Huang et al., Plant Mol. Biol. 33:125-139, 1996), Cat3 from Arabidopsis (GenBank No. U43147, Zhong et al., Mol. Gen. Genet. 251:196-203, 1996), the gene encoding stearoyl-acyl carrier protein desaturase from Brassica napus (Genbank No. X74782, Solocombe et al., Plant Physiol. 104:1167-1176, 1994), GPc1 from maize (GenBank No. X15596, Martinez et al., J. Mol. Biol. 208:551-565, 1989), Gpc2 from maize (GenBank No. U45855, Manjunath et al., Plant Mol. Biol. 33:97-112, 1997), other transcription initiation regions from various plant genes known to those of skill. See also Holtorf, “Comparison of different constitutive and inducible promoters for the overexpression of transgenes in Arabidopsis thaliana,” Plant Mol. Biol. 29:637-646, 1995).

### Inducible Promoters

Alternatively, a plant promoter may direct expression of the nucleic acids under the influence of changing environmental conditions or developmental conditions. Examples of environmental conditions that may effect transcription by inducible promoters include anaerobic conditions, elevated temperature, drought or other environmental stress, or the presence of light. Example of developmental conditions that may effect transcription by inducible promoters include senescence and embryogenesis. Such promoters are referred to herein as “inducible” promoters. For example, the invention can incorporate drought-specific promoter such as the drought-inducible promoter of maize (Busk et al., Plant J, 11: 1285-95, 1997); or alternatively the cold, drought, and high salt inducible promoter from potato (Kirch Plant Mol. Biol. 33:897-909, 1997).

Suitable promoters responding to biotic or abiotic stress conditions include the pathogen inducible PRP1-gene promoter (Ward et al., Plant. Mol. Biol. 22:361-366, 1993), the heat inducible hsp80-promoter from tomato (U.S. Pat. No. 5,187,267), cold inducible alpha-amylase promoter from potato (PCT Publication No. WO 96/12814) or the wound-inducible pinII-promoter (European Patent No. 375091). For other examples of drought, cold, and salt-inducible promoters, such as the RD29A promoter, see, e.g., Yamaguchi-Shinozalei et al., Mol. Gen. Genet. 236:331-340, 1993.

Alternatively, plant promoters which are inducible upon exposure to plant hormones, such as auxins, are used to express MG synthase genes. For example, the invention can use the auxin-response elements E1 promoter fragment (AuxREs) in the soybean (Glycine max L.) (Liu, Plant Physiol. 115:397-407, 1997); the auxin-responsive Arabidopsis GST6 promoter (also responsive to salicylic acid and hydrogen peroxide) (Chen, Plant J. 10: 955-966, 1996); the auxin-inducible parC promoter from tobacco (Sakai, 37:906-913, 1996); a plant biotin response element (Streit, Mol. Plant Microbe Interact. 10:933-937, 1997); and, the promoter responsive to the stress hormone abscisic acid (Sheen, Science 274:1900-1902, 1996).

Plant promoters inducible upon exposure to chemicals reagents that may be applied to the plant, such as herbicides or antibiotics, are also useful for expressing the MG synthase gene. For example, the maize Int-2 promoter, activated by benzenesulfonamide herbicide safeners, can be used (De Veylder, Plant Cell Physiol. 38:568-577, 19997); application of different herbicide safeners induces distinct gene expression patterns, including expression in the root, hydathodes, and the shoot apical meristem. A MG synthase coding sequence can also be under the control of, e.g., a tetracycline-inducible promoter, such as described with transgenic tobacco plants containing the Avena sativa L. (oat) arginine decarboxylase gene (Masgrau, Plant J. 11:465-473, 1997); or, a salicylic acid-responsive element (Stange, Plant J. 11:1315-1324, 1997; Uknes et al., Plant Cell 5:159-169, 1993); Bi et al., Plant J. 8:235-245, 1995).

Examples of useful inducible regulatory elements include copper-inducible regulatory elements (Mett et al., Proc. Natl. Acad. Sci. USA 90:4567-4571, 1993); Furst et al., Cell 55:705-717, 1988); tetracycline and chlor-tetracycline-inducible regulatory elements (Gatz et al., Plant J. 2:397-404, 1992); Röder et al., Mol. Gen. Genet. 243:32-38, 1994); Gatz, Meth. Cell Biol. 50:411-424, 1995); ecdysone inducible regulatory elements (Christopherson et al., Proc. Natl. Acad. Sci. USA 89:6314-6318, 1992; Kreutzweiser et al., Ecotoxicol. Environ. Safety 28:14-24, 1994); heat shock inducible regulatory elements (Takahashi et al., Plant Physiol. 99:383-390, 1992; Yabe et al., Plant Cell Physiol. 35:1207-1219, 1994; Ueda et al., Mol. Gen. Genet. 250:533-539, 1996); and lac operon elements, which are used in combination with a constitutively expressed lac repressor to confer, for example, IPTG-inducible expression (Wilde et al., EMBO J. 11:1251-1259, 1992). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

### Tissue-Specific Promoters

Alternatively, the plant promoter may direct expression of the MG synthase gene in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue. Reproductive tissue-specific promoters may be, e.g., ovule-specific, embryo-specific, endosperm-specific, integument-specific, seed and seed coat-specific, pollen-specific, petal-specific, sepal-specific, or some combination thereof.

Other tissue-specific promoters include seed promoters. Suitable seed-specific promoters are derived from the following genes: MAC1 from maize (Sheridan, Genetics 142:1009-1020, 1996); Cat3 from maize (Abler, Plant Mol. Biol. 22:10131-1038, 1993); vivparous-1 from Arabidopsis (Genbank No. U93215); atmycl from Arabidopsis (Urao, Plant Mol. Biol. 32:571-57, 1996; Conceicao, Plant 5:493-505, 1994); napA from Brassica napus Josefsson, JBL 26:12196-1301, 1987); and the napin gene family from Brassica napus (Sjodahl, Planta 197:264-271, 1995).

A variety of promoters specifically active in vegetative tissues, such as leaves, stems, roots and tubers, can also be used to express polynucleotides encoding MG synthase polypeptides. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarin promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are kn1-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, the MG synthase polynucleotide is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai, Proc. Natl. Acad. Sci. USA 92:1679-1683, 1995); the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer, Plant Mol. Biol. 31:1129-1139, 1996).

A vector comprising MG synthase nucleic acid sequences will typically comprise a marker gene that confers a selectable phenotype on the cell to which it is introduced. Such markers are known. For example, the marker may encode antibiotic resistance, such as resistance to kanamycin, G418, bleomycin, hygromycin, and the like.

MG synthase nucleic acid sequences of the invention are expressed recombinantly in plant cells as described. As appreciated by one of skill in the art, expression constructs can be designed taking into account such properties as codon usage frequencies of the plant in which the MG synthase nucleic acid is to be expressed. Codon usage frequencies can be tabulated using known methods (see, e.g., Nakamura et al. Nucl. Acids Res. 28:292, 2000). Codon usage frequency tables are available in the art (e.g., from the Codon Usage Database at the internet site www.kazusa.or.jp/codon/.)

Additional sequence modifications may be made that are also known to enhance gene expression in a plant. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence may also be modified to avoid predicted hairpin secondary mRNA structures.

### Production of Transgenic Plants

As detailed herein, the present invention provides for transgenic plants comprising recombinant expression cassettes either for expressing heterologous MG synthase proteins in a plant or for expressing GT55 family members and in some embodiments, GT55 family members and a phosphatase. It should be recognized that transgenic plants encompass the plant or plant cell in which the expression cassette is introduced as well as progeny of such plants or plant cells that contain the expression cassette, including the progeny that have the expression cassette stably integrated in a chromosome.

A recombinant expression vector comprising a MG synthase coding sequence driven by a heterologous promoter may be introduced into the genome of the desired plant host by a variety of conventional techniques. For example, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. While transient expression of MG synthase is encompassed by the invention, generally expression of construction of the invention will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described, e.g., in Paszkowski et al., EMBO J. 3:2717-2722, 1984. Electroporation techniques are described, e.g., in Fromm et al. Proc. Natl. Acad. Sci. USA 82:5824, 1985). Ballistic transformation techniques are described, e.g., in Klein et al. Nature 327:70-73, 1987).

Agrobacterium tumefaciens-mediated transformation techniques, including disarming and use of binary vectors, are well described in the scientific literature (see, e.g., Horsch et al. Science 233:496-498, 1984), and Fraley et al. Proc. Natl. Acad. Sci. USA 80:4803, 1983).

Transformed plant cells derived by any of the above transformation techniques can be cultured to regenerate a whole plant that possesses the transformed genotype and thus the desired phenotype such as enhanced drought-resistance. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally, e.g., in Klee et al. Ann. Rev. of Plant Phys. 38:467-486, 1987.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The techniques described herein for obtaining and expressing MG synthase nucleic acid sequences in plant cells can also be employed to express nucleic acid sequences that encode GT55 family members and phosphatases.

The expression cassettes of the invention can be used to confer drought resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. In some embodiments, the plant is corn, switchgrass, sorghum, miscanthus, sugarcane, poplar, pine, wheat, rice, soy, cotton, barley, turf grass, tobacco, potato, bamboo, rape, sugar beet, sunflower, willow, and eucalyptus. In further embodiments, the plant is reed canarygrass (Phalaris arundinacea), Miscanthus x giganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, or Kentucky bluegrass among others. In some embodiments, the plant is an ornamental plant. In some embodiment, the plant is a vegetable- or fruit-producing plant.

The plants of the invention have enhanced MG levels compared to plants that are otherwise identical except for expression of MG synthase; or a G55 family member and appropriate phosphatase. MG levels can be determined directly or indirectly. For example, in some embodiments, activity of MG synthase activity is assessed using enzymatic assays (see, e.g., Empadinhas, et al. J. Bacteriol. 186:4075-4084, 2004) as an indicator of MG levels. In some embodiments, the level of MG is monitored directly (e.g., Empadinhas et al, supra) or by observing or measuring a phenotype mediated by increased MG, such as increased drought tolerance, or increased tolerance to other environmental stresses.

Drought resistance can assayed according to any of a number of well-known techniques. For example, plants can be grown under conditions in which less than optimum water is provided to the plant. Drought resistance can be determined by any of a number of standard measures including turgor pressure, growth, yield, and the like.

Stress may also be evaluated using other known techniques. For example, salt tolerance in plants may be analyzed by exposing a plant to high salt, e.g., 100 mM to 600 mM NaCl, for a desired period of time, e.g., anywhere from 1 hour to 7 days. Plants are then allowed to grow, for example from 1 to 3 weeks at an appropriate temperature and survival rate is determined. Low-temperature stress can be evaluated, for example, by exposing a plant that is recognized as being a lower temperature for that plant, e.g., temperature ranging from −15° C. to 5° C. for a period of time, for example, ranging from 30 minutes to 10 days, growing the plants for a period of time following cold exposure, for example for 2 days to 3 weeks at 20° C. to 35° C., and then examining the survival rate. Heat tolerance may also be evaluated by exposing a plant to an elevated temperature for a period of time and evaluating survival.

In some embodiments, accumulation of mannosylglycerate is in addition to, i.e., “stacked on top of”, accumulation of an endogenous compatible solute where the latter solute is not undesired. For example, the disaccharide trehalose is a compatible solute commonly used by seed plants that typically has no unwanted side effects. Accordingly, in one embodiment, MG accumulation is in addition to trehalose accumulation. In some embodiments, trehalose and MG may have a synergistic effect on carbohydrate metabolism.

In some embodiments, a plant is engineered to produce MG that has also been engineered to prevent, or lower, solute accumulations where the solute is undesirable. For example, in one embodiment, the plant is selected among species and cultivars that already accumulate a compatible solute where the solute is glycine betaine, which is found in various species, including, but not limited to, beet, spinach, wheat, barley, corn, sugarcane, sunflower and cotton. Glycine betaine lowers the value of the biomass as feed, interferes with some industrial processing steps (e.g., sucrose crystallization), serves as attractant of some pests and pathogens, and as there is no catabolic fate of glycine betaine in higher plants, it is also undesired in biofuel crop plants. A replacement strategy in which the native compatible solute is replaced with MG is thus attractive in a number of situations. Workers skilled in the art will understand that there are several ways to reduce, or eliminate, accumulation of the native compatible solute: Traditional selection breeding techniques may be employed to select for plants that have reduced accumulation of native compatible solutes. Other techniques to reduce or eliminate native compatible solute accumulation include; antisense or RNA interference techniques that inhibit expression of an essential enzyme—choline monooxygenase for example in the case of glycine betaine. In corn, a single gene mutant is known that knocks out glycine betaine accumulation. Thus, for example, introducing MG accumulation in this corn mutant can results in more drought resistant corn that accumulates a solute that has a catabolic fate in the plant and is fermentable (biofuel) or metabolizable (feed) post harvest.

A practitioner in this art will also understand that MG synthesis can not only be controlled by promoters such as generally stress responsive promoters, but may also be operably linked to promoters that regulate accumulation of the compatible solute that is replaced, e.g., promoters that are involved in glycine betaine accumulation, trehalose accumulation and the like.

The replacement technology is not limited to glycine betaine. One of the most drought resistant crop plants know is the grasspea, Lathyrus sativus. It uses the non-protein amino acid, 2-amino-2-carboxyethyloxamid acid, as compatible solute. This amino acid is a neurotoxin causing the crippling disorder neurolathyrism. Traditional breeding has yielded grasspea cultivars free of the neurotoxin, but not without sacrificing some of the extreme tolerance to temporary drought. Accordingly, grasspea may also be engineered to produce MG, preferably in a cultivar that lacks, or has reduced, 2-amino-2-carboxyethyloxamid acid accumulation.

The foregoing and other aspects of the invention may be better understood in connection with the following non-limiting examples.

### EXAMPLES

### Example 1

### Expression of MG Synthase

The sequence of the mannosylglycerate synthase gene from Selaginella moellendorffii GT78A1 is shown in SEQ ID NO:5. The sequence was codon optimized for its expression in plants. SEQ ID NO:7 provide an example of a codon-optimized sequence that was used in the present examples, but it is generally appreciated in the art that there are many other codon-optimized alternatives that can be used based on the following: Codon use satisfies the criteria for translation in the host cell and the change in codon use does not introduce new secondary structures in the transcript. SEQ ID NO:7 was synthesized by Genscript and inserted into a suitable vector. Many vectors are known that can be used. In this example, pUC57, was employed.

The heat shock promoter (hsp81) (SEQ ID NO:9) was amplified using the following primers: forward primer containing a SacI site in the 5′end 5′ GGGGAGCTCGATATCGGTTTGAAGATGGCAAGTG 3′ (SEQ ID NO:10) and reverse primer containing a KpnI site on the 3′ end (shown 5′ to 3′ orientation 5′ GGGGTACCATCGCAACGAACTTTGATTCAACGC 3′(SEQ ID NO:27)). The PCR program used was the following: 94° C. for 3 min. initial denaturation then 94° C. for 30 sec., 52° C. for 30 sec., 72° C. for 45 sec, 30 cycles in total.

The sequence of the ubiquitin promoter (ubi) SEQ ID NO:13 was amplified using the following primers: forward primer containing a SacI site in the 5′end 5′ GGGGAGCTCACCTGCAGAAGTAACACCAAACAACAG 3′ (SEQ ID NO:14) and reverse primer containing a KpnI site on the 3′ end GGGAAGCTTCTGCAGTGCAGCGTGACCCGGTCGTG (SEQ ID NO:15). The PCR program used was the following 94° C. for 3 min initial denaturation, then 94° C. for 30 sec, 52° C. for 30 sec, 72° C. for 75 sec, 30 cycles in total.

The amplified promoters were digested with the Sad and KpnI restriction enzymes and ligated into the vector with the GT78A1 sequence, the vector also digested with the same enzymes. The ligation mix was used to transform E. coli. Ampicilin-resistant colonies were selected and grown in 5 mL overnight cultures to prepare a DNA miniprep. Vectors containing the desired hsp81-GT78A1 or ubi-GT78A1 chimeric constructs were quality controlled using the M13 rev primer and the promoter reverse primer as sequencing primers.

To insert the GT78A1 linked to the desired promoter in a plant transformation vector, the following strategy was used. The hsp81-GT78A1 fragment was amplified by PCR here given for the hsp81-GT78A1 vector as template and the following primers: forward primer containing a HindIII site in the 5′end GGGAAGCTTGATATCGGTTTGAAGATGGCAAGTG (SEQ ID NO:12) and reverse primer containing a BstEII site on the 3′ end GGGGGTNACCTCAAACAGCACATGCAGCATCCATC (SEQ ID NO:8). The PCR program used was the following 94° C. for 3 min. initial denaturation then 94° C. for 30 sec, 52° C. for 30 sec, 72° C. for 3.5 min, 30 cycles in total. The ubi-GT78A1 fragment was amplified by PCR using the ubi-GT78A1 vector as template and the following primers: forward primer containing the HindIII site in the 5′end GGGAAGCTTCCTGCAGAAGTAACACCAAACAACAG (SEQ ID NO:16) and reverse primer containing the BstEII site on the 3′ end GGGGGTNACCTCAAACAGCACATGCAGCATCCATC (SEQ ID NO:8). The PCR program used was the following 94° C. for 3 min initial denaturation, then 94° C. for 30 sec, 52° C. for 30 sec, 72° C. for 3. 5 min, 30 cycles in total.

The plant transformation vector pCAMBIA2301 (www.cambia.org) was digested with HindIII and BstEII restriction enzymes and gel purified in order to remove the lacZ-35S-GUS fragment from the vector. The fragments hsp81-GT78A1 and ubi-GT78A1 were digested with HindIII and BstEII restriction enzymes and ligated into the digested and purified pCAMBIA2301. The ligation mix was used to transform E. coli DH5α strain. Kanamycin-resistant colonies were selected and grown in 5 mL overnight cultures to prepare DNA miniprep. Vectors containing the desired promoter pCAMBIA-hsp81-GT78A1 and pCAMBIA-ubi-GT78A1 were quality controlled using pCAMBIA2301-F (CTAGAGTCGACCTGCAGGCATGC (SEQ ID NO:28)) primer and pCAMBIA2301-R (CGATCGGGGAAATTCGAGCTG (SEQ ID NO:29)) primer as sequencing primers. The pCAMBIA-hsp81-GT78A1 and pCAMBIA-ubi-GT78A1 were then introduced into Agrobacterium tumefaciens AGL1 strain by electroporation for further Brachypodium distachyon plant transformation.

For expression with 35S promoter, the coding region of the synthetic gene was amplified by PCR with forward primers CACCATGTCTCTTGTTTGTTT (SEQ ID NO:30) and CACCTCTCTTGTTTGTTTTCC (SEQ ID NO:31), for expression without or with N-terminal HA-fusion tag. The reverse primer was TCAAACAGCACATGCAG (SEQ ID NO:32). The PCR program used was the following: 98° C. for 30 sec initial denaturation, then 98° C. for 10 sec, 60° C. for 30 sec, 72° C. for 45 sec, 35 cycles in total, final extension 72° C. for 10 min. The polymerase used was Phusion (Finnzymes).

As understood in the art, many vector systems for cloning are available. In this example, the cloning system employed Gateway and Topo vectors, however, alternative cloning systems could also have been used. The PCR products were cloned into pENTR/SD/D-TOPO (Invitrogen). The inserts were transferred into Gateway (Invitrogen) destination vectors by LR reaction. The vector pEarleyGate 100 was used for expression of the unmodified protein and pEarleyGate 201 was used for expression of the protein with an N-terminal HA (hemagglutinin) tag. The vector for rice transformation was made by LR cloning using the same entry clone and an Ubi-NC1300RFCA destination vector (Peng et al. 2008). The pEarlyGate100-35S-GT78A1, pEarlyGate201-35S-HA-GT78A1, and pUbi-GT78A1-NC1300RFCA vector constructs were confirmed by DNA sequencing.

### Example 2

### Transformation of Arabidopsis and Tobacco Plants

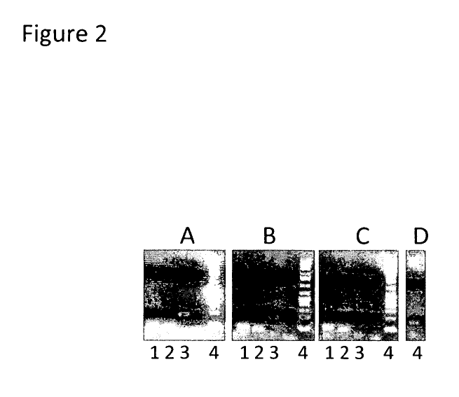

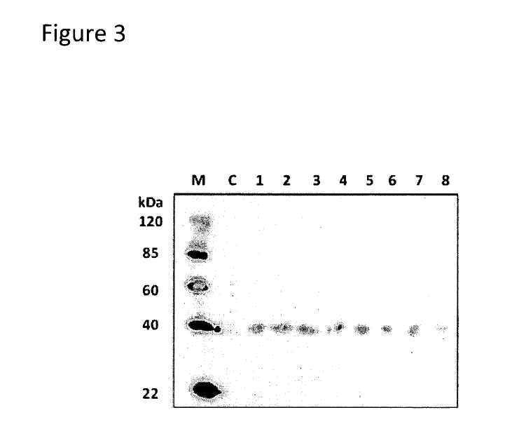

The constructs with 35S and heat shock promoters were transformed into Agrobacterium tumefaciens and used for Agrobacterium mediated transformation of plants. Arabidopsis thaliana plants were transformed by the floral dip method with all three types of construct and Nicotiana benthamiana leaves were infiltrated with Agrobacterium carrying the pEarleyGate 201 construct according to Voinnet et al. (2003) for transient expression of the HA-tagged fusion protein. As understood in the art any number of different vectors can be employed. The vector pEarleyGate 201 is one example. Arabidopsis T1 transformants were selected by spraying with Basta and the presence of the transgene confirmed by PCR. Infiltrated Nicotiana plants were grown for 3 to 4 days before analysis for expression of the MG synthase. Expression of the gene in Arabidopsis was tested by RT-PCR (FIG. 2). Presence of the HA-tagged fusion protein in Nicotiana was confirmed by Western blotting using an antibody against the HA tag (FIG. 3). Arabidopsis plants transformed with the construct for HA-tagged fusion protein were likewise confirmed by Western blotting to express the MG synthase (data not shown).

### Example 3

### Production of MG in Transgenic Plants

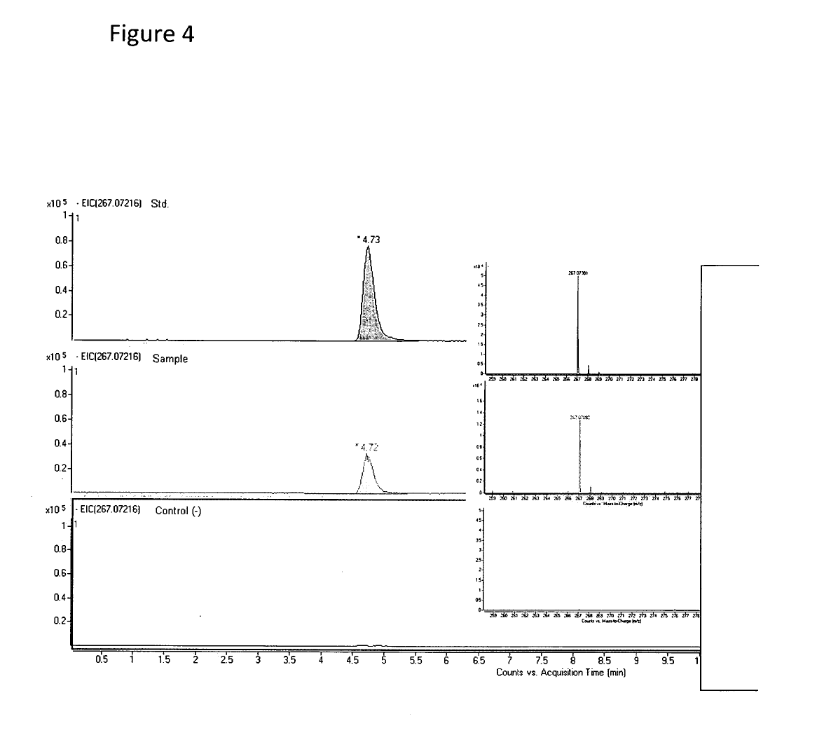

Nicotiana plants expressing the HA-tagged MG synthase were grown for 3-4 days after infiltration before leaves were harvested. Arabidopsis stable transformants expressing either the unmodified MG synthase or the HA-tagged fusion protein under control of 35S promoter were grown for 4-8 weeks before rosette leaves were harvested. Leaves (20-100 mg) were frozen in liquid nitrogen and crushed to a powder using a metal ball shaker. Shaking was at 20 Hz for 1-2 min. The leaf powder was added 1 ml of chloroform/methanol 1:1 and 250 μl of water. The sample was thoroughly vortexed, and was let to stand at 4° C. for 30 min before centrifugation at 10000×g for 5 min at 4 C. The aqueous (upper) phase was transferred to a new tube and 0.75 ml of chloroform was added. The sample was vortexed and centrifuged. The aqueous phase was transferred to a new tube and extracted again with 0.6 ml of chloroform and centrifuged. The aqueous phase was filtered in a spin filter and stored frozen until analysis by LC-MS. The extracts were analyzed by LC-MS an Aminex, Fermentation Monitoring column, 150×7.8 mm (Bio-Rad). MG was shown to be present in all the transgenic plants tested but no MG could be detected in untransformed Nicotiana and Arabidopsis plants. FIG. 4 shows an example of LC-MS data confirming the presence of MG in transformants. In some experiments, internal standards of MG were added to the leaves during the extraction procedure. These experiments confirmed that no significant amount of MG was lost during the procedure.

### Example 4

### Transformation of Grasses with MG Synthase

Transformation of Brachypodium distachyon. Immature seeds were harvested when most of the seeds were starting to fill out. Whole seed heads were harvested into tubes and the cap was kept on to prevent seeds to dry out. The lemna was removed manually. The remaining part was surface sterilized with 10% sodium clorite with 1 drop tween per 10 ml for 4 minutes with shaking and rinsed with sterile water three times. The palea was removed using fine forceps and a dissecting microscope under sterile conditions. Embryos that were clear and smaller than 1 mm were chosen to give the best frequency of embryogenic calli. The embryos were placed with the scutellar side down on CIM media in 9 cm Petri dishes. CIM medium was composed of (4.43 g/l LS-salts and 30 g/l sucrose, adjusted to pH 5.8 prior to addition of 2 g/1 Phytagel and autoclaving. After autoclaving, 0.5 ml of a 5 mg/l 2,4-D solution and 1 ml of a 0.6 mg/l CuSO₄ solution were added. The plates were sealed with parafilm and incubated at 26° C. in the darkness. After 3 weeks the embryogenic structure was divided into smaller pieces and transferred to new CIM plates. After another 2 weeks the embryos were divided again and transferred to new CIM media still kept at 26° C. in the darkness.

After 1 additional week the embryogenic calli were ready to transform with Agrobacterium.

Two to four days before co-cultivation, the Agrobacterium strain was streaked out on LB medium plates with the appropriate antibiotics. The Agrobacterium suspension was made by scraping Agrobacterium off the plates and re-suspending in liquid CIM medium (without CuSO₄) to a density of OD₆₀₀=0.6. Acetosyringone was added to a final concentration of 200 μM and 10 uL 10% Synperonic PE/F68 (Sigma, old name Pluronic F68) per 1 mL Agrobacterium was added to the suspension medium.

The 50-100 embryonic calli were added to 10-15 ml of the Agrobacterium suspension and left for 5 minutes. In the meantime, one piece (each) of 8 cm sterile whatman filterpaper was placed in empty Petri dishes. The inoculated embryogenic calli and the Agrobacterium suspension was poured into a Petri dish and all excess liquid removed with a sterile plastic Pasteur pipette. A reasonable lump (app. 50 mg) of embryogenic calli were placed on the dry filter paper filled Petri dishes and spread evenly. The plates were incubated at 22° C. in the darkness for 3 days.

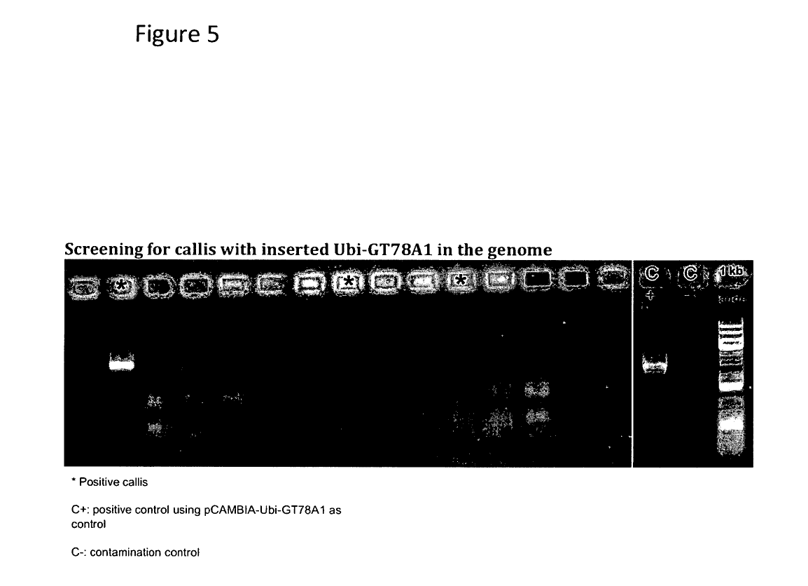

Confirmation of the transgenic state of Brachypodium Cells Half of a callus was used from a sterile culture to check for the insertion if the hsp81-GT78A1 or ubi-GT78A1 into Brachypodium distachyon genome. Genomic DNA was extracted according to Kasijima et al. (2004). 2 uL of the extracted DNA was used as template for PCR analysis of a final volume of 20 uL. The primer combination used to detect the ubi-GT78A1 fragment was: forward primer containing a HindIII site in the 5′end GGGAAGCTTCCTGCAGAAGTAACACCAAACAACAG (SEQ ID NO:16) and reverse primer containing a BstEII site on the 3′ end GGGGGTNACCTCAAACAGCACATGCAGCATCCATC (SEQ ID NO:8). The PCR program used was the following 94° C. for 5 min. initial denaturation then 94° C. for 30 sec., 52° C. for 30 sec., 72° C. for 3min. 30 sec, 30 cycles in total. 6 callis out of 94 presented a 3 kb band corresponding to the size of the ubi-GT78A1 fragment, see FIG. 5.

Rice was transformed with Agrobacterium mediated transformation using the pUbi-GT78A1-NC1300RFCA vector. Transformation of rice was according to standard procedures well known to those skilled in the art and essentially as described (Chern et al. 2001). Transgenic calli were selected on hygromycin containing medium.

### Example 5

### Regeneration of Plants from Transgenic Calli

Rice plants were regenerated from transgenic calli as described (Chern et al. 2001). Leaves of transgenic rice plants were extracted in the same way as described for Arabidopsis and tobacco leaves, and tested for the presence of MG by LC-MS. MG was detected in transgenic rice plants, while no MG was detected in untransformed control plants.

The embryogenic Brachypodium calli of Example 4 were then placed on plates with CIM media with 200 mg/l Timentin and the appropriate selection marker. The plates were incubated at 26° C. in the darkness for one week and then transferred to the same media again. After another 2 weeks, a few of the embryogenic calli had started to grow as sign of transformation. The embryogenic calli were transferred to new CIM media with 200 mg/l Timentin and the appropriate selection marker. Only those with new growth were transferred to regeneration media with 200 mg/l Timentin and the appropriate selection marker and incubated at 22° C. under a light regime of 16 hours light and 8 hours darkness. The embryogenic calli on regeneration medium (RM, prepared as CIM medium without CuSO₄ and with 0.2 mg/l kinetin as the hormone supplement rather than 2,4D media) were transferred to new RM media with 200 mg/l Timentin and the appropriate selection marker every 2-3 weeks. After four weeks 1% of the calli produced new plantlets with shoots emerging thereafter. When shoots are large enough they are transferred to MS media in bigger containers eventually to be transplanted to soil.

One of skill understands that modifications to the transformation procedure can be made based on the line of Brachypodium employed. Further, alternative transformation procedures known in the art may be employed where desired based on the line of Brachypodium that is used. Several examples of protocols for transformation and regeneration of Brachypodium plants are found in the references (Christiansen et al. 2005; Vogel et al. 2006, 2008; Vain et al. 2008; Pacurar et al. 2008; Bablak et al. 1995; Draper et al. 2001).

### Example 6

### Expression of Bifunctional Mannosyl-Phospholgycerate Synthase/Mannosyl-Phospho-Glycerate Phosphatase

In another example, plants are genetically modified to express the bifunctional mannosyl-phospho-glycerate synthase/mannosyl-phospho-glycerate phosphatase (Empadinhas et al. 2004) from Dehalococcoides ethenogenes with a plastid transit peptide. Expression of the enzyme has been shown in yeast to result in MG accumulation (Empadinhas et al. 2004). Plants expressing trehalose synthase (Garg et al. 2002, Karim et al. 2007) are used for reference. Alternatively, plants can be transformed with separate genes for mannosyl-phosphate-synthase and mannosyl-phospho-glycerate phosphatase which are present in several different microorganisms and fungi.

### Example 7

### Accumulation of MG and Other Compatible Solutes Under Different Growth Conditions

The transgenic plants are tested for the accumulation of MG and naturally occurring compatible solutes using LC-MS under normal growth conditions and in response to heat stress, drought stress and salt stress. Recovery from temporary stress and the fate of the MG during recovery are likewise assessed. Further analyses include an assessment of crop yield (biomass and grain yield) under stress conditions of different duration. Furthermore, trehalose has a regulatory effect on carbohydrate metabolism (Paul et al. 2008) and there are indications that trehalose and MG have a synergistic effect (Santos and da Costa 2002). The results of combining stress-induced accumulation of trehalose and MG are also assessed.

### REFERENCES

Borges N, Ramos A, Raven N D H, Sharp R J, Santos H (2002) Comparative study on the thermostabilising effect of mannosylglycerate and other compatible solutes on model enzymes. Extremophiles 6: 209-216.
Empadinhas N, Albuquerque L, Costa J, Zinder S H, Santos M A S, Santos H, da Costa M S (2004) A gene from the mesophilic bacterium Dehalococcoides ethenogenes encodes a novel mannosylglycerate synthase. J Bacteriol 186: 4075-4084.
Garg A K, Kim J-K, Owens T G, Ranwala A P, Choi Y D, Kochian L V, Wu R J (2002) Trehalose accumulation in rice plants confers high tolerance levels to different abiotic stresses. Proc Natl Acad Sci USA 99: 15898-15903
Karim S, Aronsson H, Ericson H, Pirhonen M, Leyman B, Welin B, Mäntylä E, Palva E T, van Dijck P, Holmström K-O (2007) Improved drought tolerance without undesired side effects in transgenic plants producing trehalose. Plant Mol Biol 64:371-386
Paul M J, Primavesi L F, Jhurreea D, Zhang Y (2008) Trehalose Metabolism and Signaling. Annu Rev Plant Biol 59:417-41
Penna S (2003) Building stress tolerance through over-producing trehalose in transgenic plants. Trends Plant Sci 8: 355-357
Santos H, da Costa M S (2002) Compatible solutes of organisms that live in hot saline environments. Env Microbiol 4: 501-509
Santos D M, Torné J M, Fevereiro P S, Almeida A M, Cardoso L A (2007) Trehalose and its applications in plant biotechnology. In Vitro Cell Dev Biol Plant 43:167-177
Schluepmann H, van Dijken A, Aghdasi M, Wobbes B, Paul M, Smeekens S (2004) Trehalose mediated growth inhibition of Arabidopsis seedlings is due to trehalose-6-phosphate accumulation. Plant Physiol 135:879-890
Silvia C Alves, Barbara Worland, Vera Thole, John W Snape, Michael W Bevan & Philippe Vain, A protocol for Agrobacterium-mediated transformation of Brachypodium distachyon community standard line Bd21, Nature Protocols, vol 4, no 5, pp 638-649 (2009)
Chern M S, Fitzgerald H A, Yadav R C, Canlas P E, Dong X, Ronald P C (2001). Evidence for a disease-resistance pathway in rice similar to the NPR1-mediated signaling pathway in Arabidopsis. Plant J. 27: 101-113.
Voinnet O, Rivas S, Mestre P, Baulcombe D (2003) An enhanced transient expression system in plants based on suppression of gene silencing by the p19 protein of tomato bushy stunt virus. Plant J 33: 949-956
Christiansen, P., Andersen, C. H., Didion, T., Folling, M. & Nielsen, K. K. A rapid and efficient transformation protocol for the grass Brachypodium distachyon. Plant Cell Rep. 23, 751-758 (2005).
Vogel, J. P., Garvin, D. F., Leong, O. M. & Hayden, D. M. Agrobacterium-mediated transformation and inbred line development in the model grass Brachypodium distachyon. Plant Cell Tissue Organ Cult. 84, 199-211 (2006).
Vogel, J. & Hill, T. High-efficiency Agrobacterium-mediated transformation of Brachypodium distachyon inbred line Bd21-3. Plant Cell Rep. 27, 471-478(2008).
Vain, P. et al. Agrobacterium-mediated transformation of the temperate grass Brachypodium distachyon (genotype Bd21) for T-DNA insertional mutagenesis. Plant Biotechnol. J. 6, 236-245 (2008).
Pacurar, D. I., Thordal-Christensen, H., Nielsen, K. K. & Lenk, I. A high-throughput Agrobacterium-mediated transformation system for the grass model species Brachypodium distachyon L. Transgenic Res. 17, 955-963 (2008).
Bablak, P., Draper, J., Davey, M. R. & Lynch, P. T. Plant regeneration and micropropagation of Brachypodium distachyon. Plant Cell Tissue Organ Cult. 42, 97-107 (1995)
John Draper, Luis A. J. Mur, Glyn Jenkins, Gadab C. Ghosh-Biswas, Pauline Bablak, Robert Hasterok, and Andrew P. M. Routledge, Brachypodium distachyon. A new model system for functionalgenomics in grasses. Plant Physiol. 127, 1539-1555 (2001).
Kasijima I, Ide Y, Ohkama-Ohtsu N, Hayashi H, Yoneyama T, Fujiwara T (2004) A protocol for rapid DNA extraction from Arabidopsis thaliana for PCR analysis. Plant Molecular Biology Reporter 22: 49-52
Peng Y, Bartley L E, Chen X, Dardick C, Chern M, Ruan R, Canlas P E, Ronald P C (2008) OsWRKY62 is a Negative Regulator of Basal and Xa21-Mediated Defense against Xanthomonas oryzae pv. oryzae in Rice. Molecular Plant 1, 446-458.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications, patent documents, and accession number cited herein are incorporated herein by reference as if each such publication, document, or accession number was specifically and individually indicated to be incorporated herein by reference. Citation of publications and patent documents is not intended as an indication that any such document is pertinent prior art, nor does it constitute any admission as to the contents or date of the same.

### Additional Mannosylglycerate Synthase Sequences and Sequences Related to Examples:

```
bifunctional mannosylglycerate synthase Dehalococcoides ethenogenes
SEQ ID NO: 4
mrieslrpae hlgnvtihgv rrfleldsga pslphlqesr evemvdqqai sdvekkmaii
lpikdedlkv fegvlsgiph dclmivisns skqevdnfkn ekdivsrfcr ithrgaivvh
qknpelagai adagypellg kdglirsgka egmilgiilt mfsgreyvgf idtdnyipga
vweyakhfat gfnlaqspys mvrvlwkykp klvgdlyfkr wgrvsevtnk hlnhlisskg
kfeteiiktg nagehamsie lakrltygtg yavetkeims ileqfsgilp iedrevaeeg
veilqtetin phlhedkggd hliqdmllps laviyhspla desvkkkien qlagieglen
npeipqvkli pppqkmdlak fsaviekylp qmvlpdgeli sriarpsrlp ssgqfkkviy
tdldgtllsp ltysystald alrllkdkel plvfcsaktm geqdlyrnel gikdpfiten
ggaifipkdy frlpfaydrv agnylvielg msykdirhil kkalaeacte ienseragni
fitsfgdmsv edvsrltdln lkqaelakqr eysetvhieg dkrstnivln hiqqngleys
fggrfyevtg gndkgkaikv lnelfrlnfg nihtfglgds endysmletv dspilvqrpg
nkwhkmrlrn psyvrgvgpe gfsravtdii lpme
Coding sequence of Selaginella moellendorfii MG synthase
SEQ ID NO: 5
      1 ATGTCGCTGGTGTGCTTTCCATTCAAGGAGGAGGACGTTGCCGTGGTCGTCCGCAACGTAGAGTGCGCCG 70
    71 CTGCTCACCCGCGAGTCTCCACCGTGCTGTGCGTCGGCTACAGCAAAGGCGAGACGTGGTGCGCCATCGA 140
  141 GGCCAAGAGACCGTCCATCGAGAGCTCCACCGGCAAGCGCATCATCCTCCTCCAGCAGAAGCGCATCGGG 210
  211 GTGTCGCTGCGATCCGGCAAAGGCGATGGAATGAACACGGCGCTGGCCTACTTTCTCGACCACACAGAGC 280
  281 TCAAGCGCATCCATTTCTACGACTCCGACATCGTCTCCTTCTCCGCGGACTGGATCACCAAGGCCGAGCG 350
  351 GCAGGCGGATCTAGACTTCGACGTGGTCCGGCACTACTTCCCCAGATCCAGCACCGACGCTATGATCACT 420
  421 TGGTTTGTAACGAAGATCGGTTTTTGCCTCCTCTGGCCCAAGACGGTGCTGCCATTCATCGAGCAGCCAC 490
  491 TGGGCGGAGAGCTCCTACTCACGAGAAAGGCCGCCGAGGCGCTCTACACCGATCACCGAGTTCGCGGCCA 560
  561 GAGTGACTGGGGCATCGACACTCTCTACACTTTCATCATGGTCCAGAAGGGTCTCCACTTGGCCGAGGTC 630
  631 TACATCCCGGAGGGCAAGGTCCACGCCCTCTACTCGGGCCTTCGGGATCTAAGGACCATGCTGGTGGAGT 700
  701 GCTTCTCGGCAATGCAGTCGCTCAAGGACGAGGCGGTGCCGCTCAACGAGGGGACTCACAGGATGGAGTA 770
  771 CACCCGGCCGGTGCCCGAGCTTGTGAAGCAAAAGGTTGGCTACGACGTGGAGAAGACGCTCAAGCTCTTG 840
  841 CGGAGCAACTGGACGCAGGGGCAGCGGGATCTCCTCCAGAAGCACTTCGATCCGGCGCTCGCCAAGGGGC 910
  911 TCCTGAATGCCAGCGAGTGGCCGACGTGGGGCTTTGCCGACGAGGAGGCGTGGGTGGCGGCGTACCGGAC 980
  981 GCTCTTGGTTCACTTCGAGAAAGGTGACGAGGACTGGGAGGAGCTGCTGTTTAAGATCTGGGTGAGCAGG 1050
1051 GTACTCAACCACACCATGAGGCACTCGCTCCGGGGATACGATGCTGCTCTGGATGCTCTGAGGAGCTTGA 1120
1121 TCTGGGAGACGCAGCACCAGTCTGCGATGCTGTCCAAGAGCGCTGCTGCGAATCATCACATCGTCTCGGG 1190
1191 GCATTCAGCGTCGGAGGCTCTGCCGAGAACCGCTGGTCAGCTTCGGGAGAAACGCATGGACGCTGCCTGT 1260
1261 GCTGTTTGA 1269
Coding sequence of Physcomitrella patens MG synthase
SEQ ID NO: 6
ATGTCTTTGGTGTGCTTCCCATTCAAGGAAGAAGATGTCGCAGTGGTGGTTGGTA
ATATAGAGTGCGCAGCATCGCATCCCCGGGTTGCAGCCATTTTGTGTGTGGGCTA
CTCGCAAGGAGAGACGTGGCACGCCATTGAAGCTGCTAAGCCACGCATTGAGAG
AGCTACAGGAAAGGAAATTTTTCTGGTGCTGCAGCGACGGATTGGTGTGAGCCTT
CGTGGAGGCAAAGGTGATGGCATGAACACGGCACTAGCTTTCTTTCTGGAGAAG
GACGTGTATCCGCGTCTGCACTTCTACGACGCTGATATTGTCTCGTTCTCCGGAG
AGTGGATTTCCAAAGCAGAGAAGCAGGCGGATCTCGACTACGATATCGTCCGTC
ATTACTTCCCGAGATCCAGCACGGATGCCATGATCACATGGCTTGTGACGAAGCT
GGGGTTTGCTATGCTGTGGCCCAACTCGACTTTGCCTTTCATTGAGCAACCCCTCG
GTGGAGAACTCCTGCTAACGAAAAAGGCGGCTGAAGTGCTCTACGGAGACCACC
GTGTTCGATCTCAAAGTGACTGGGGGATAGATACTCTTTACACTTTTGTCACCGC
ACAAGCAGGTCTACGTCTCGCTGAGGTTTACATCTCTGAAGGGAAGGTCCATGCG
CTATATGGTGGTCTTCGAGATCTCCGGACTATGCTCGTGGAATGTTTCTCAGCGG
TGCAGTCGCTACGTAAGGAAATTCTTCCCAGTGAAGATGGAGCAGTTCACTGCAT
TGAACCAGCTAAGGCCGTCCCGGACTTGATCAAACAAAAAGTCGGGTACGACAT
AGAAAAGACATTGAAGCTGTTGAAGAGCAATTGGACACCTCGACAGCAGGAGCT
TCTGCACCAGCATTTTGACTCTGCCCTTGTGAAGGGATTGTTGAACTCTGCCGAG
TGGCCGTGCTGGAGTTTCGCAGATGAAGATGCCTGGACAAGCGCATATCTAAAA
TTCCTCGACCACTTCGAGAAGGGAGATAGTGATTGGGAGGAGCTTCTGTTTAAGG
TTTGGGTGGCCCGGGTACTGAACCACACTTTCAAGAATGTAATGCGTGGGTACGA
CAGTGCCCTAGGTGCTTTGCGTGACTTGGTGTGGGACACTCGCCACCAGTTTGCT
GTTAAGCTTAAAGCCAATTCCGTCCCCAACCACGCCATCGTGTCGGGCCACTCAG
CTGCAGAGGGGCTGGTCACCCGAGCTCAATCCGGCCGCAAGAAACCCAAATTCG
AGGTTGAAAATCTGTGTACAGTGCAACAGTAA
Selaginella moellendorffii GT78A1 CDS after codon optimization for plant
expression
SEQ ID NO: 7
ATGTCTCTTGTTTGTTTTCCATTCAAGGAAGAGGATGTTGCTGTTGTGGTTAGAAA
TGTGGAGTGCGCTGCAGCTCATCCTAGAGTGTCTACTGTTTTGTGTGTGGGTTATT
CAAAGGGAGAAACATGGTGCGCTATTGAGGCAAAAAGACCATCTATCGAATCTT
CAACTGGAAAGAGAATTATTCTTTTGCAACAGAAGAGAATCGGAGTTTCTCTTAG
ATCAGGAAAGGGAGATGGTATGAATACTGCTCTTGCATATTTTCTTGATCATACA
GAGTTGAAGAGAATCCATTTCTACGATTCTGATATCGTTTCTTTCTCAGCTGATTG
GATCACAAAGGCTGAAAGACAAGCAGATCTTGATTTTGATGTGGTTAGACATTAC
TTCCCTAGATCTTCAACTGATGCTATGATTACTTGGTTTGTTACAAAGATCGGATT
CTGTCTCCTTTGGCCTAAAACAGTGCTCCCATTCATTGAACAGCCTCTTGGAGGT
GAGTTGCTCCTTACTAGAAAAGCAGCTGAGGCTCTCTATACAGATCACAGAGTTA
GAGGTCAATCTGATTGGGGAATCGATACTTTGTACACTTTTATTATGGTGCAGAA
GGGTCTCCATCTTGCTGAAGTTTATATCCCAGAGGGTAAAGTGCACGCATTGTAC
TCAGGATTGAGAGATCTCAGAACTATGCTCGTTGAATGCTTCTCTGCTATGCAAT
CATTGAAAGATGAAGCAGTGCCTCTCAATGAGGGAACTCATAGGATGGAATATA
CAAGACCTGTTCCAGAGCTTGTGAAGCAAAAAGTTGGTTACGATGTGGAAAAGA
CTTTGAAATTGCTCAGATCTAACTGGACACAAGGACAGAGAGATCTTTTGCAGAA
GCATTTTGATCCAGCTCTTGCAAAAGGTCTCCTTAACGCTTCAGAGTGGCCTACTT
GGGGATTCGCTGATGAAGAGGCATGGGTTGCAGCTTATAGAACATTGCTCGTGC
ACTTTGAAAAGGGAGATGAGGATTGGGAAGAGCTTTTGTTCAAAATTTGGGTTTC
TAGAGTGCTCAATCATACTATGAGACACTCACTTAGAGGATACGATGCAGCTCTT
GATGCTTTGAGATCTCTCATTTGGGAAACACAACATCAGTCAGCTATGTTGTCTA
AGTCAGCAGCTGCAAACCATCACATCGTTTCTGGTCACTCTGCTTCAGAGGCACT
TCCTAGAACAGCTGGACAGTTGAGAGAAAAAAGGATGGATGCTGCATGTGCTGT
TTGA
Primer, Reverse-BstEII
SEQ ID NO: 8
GGGGGTNACCTCAAACAGCACATGCAGCATCCATC
Hsp81 promoter
SEQ ID NO: 9
GATATCGGTTTGAAGATGGCAAGTGTTCTTGTAATGACTATTGGTGAAGAAGACA
AATGAGAGTTGGTTTATATTTAACCATAATTTCATTCAGTTCACACTGAACCGGC
GAAATTTCTTTGCCAGACCTATTCGGAATTGAAACAAGTGGAGTCTCGAAACGAA
AAGAACTTTCTGGAATTCGTTTGCTCACAAAGCTAAAAACGGTTGATTTCATCGA
AATACGGCGTCGTTTTCAAAGAACAATCCAGAAATCACTGGTTTTCCTTTATTTC
AAAAGAAGAGACTAGAACTTTATTTCTCCTCTATAAAATCACTTTGTTTTTCCCTC
TCTTCTTCATAAATCAACAAAACAATCACAAATCTCTCGAAACGCTCTCGAAGTT
CCAAATTTTCTCTTAGCATTCTCTTTCGTTTCTCGTTTGCGTTGAATCAAAGTTCGT
TGCGAT
Primer Forward-SacI
SEQ ID NO: 10
GGGGAGCTCGATATCGGTTTGAAGATGGCAAGTG
Primer Reverse-KpnI
SEQ ID NO: 11
GGGGGTACCATCGCAACGAACTTTGATTCAACGC
Primer Forward-HindIII
SEQ ID NO: 12
GGGAAGCTTGATATCGGTTTGAAGATGGCAAGTG
Ubi promoter
SEQ ID NO: 13
ACCTGCAGAAGTAACACCAAACAACAGGGTGAGCATCGACAAAAGAAACAGTA
CCAAGCAAATAAATAGCGTATGAAGGCAGGGCTAAAAAAATCCACATATAGCTG
CTGCATATGCCATCATCCAAGTATATCAAGATCAAAATAATTATAAAACATACTT
GTTTATTATAATAGATAGGTACTCAAGGTTAGAGCATATGAATAGATGCTGCATA
TGCCATCATGTATATGCATCAGTAAAACCCACATCAACATGTATACCTATCCTAG
ATCGATCCCGTCTGCGGAACGGCTAGAGCCATCCCAGGATTCCCCAAAGAGAAA
CACTGGCAAGTTAGCAATCAGAACGTGTCTGACGTACAGGTCGCATCCGTGTACG
AACGCTAGCAGCACGGATCTAACACAAACACGGATCTAACACAAACATGAACAG
AAGTAGAACTACCGGGCCCTAACCATGGACCGGAACGCCGATCTAGAGAAGGTA
GAGAGGGGGGGGGGGGGAGGACGAGCGGCGTACCTTGAAGCGGAGGTGCCGAC
GGGTGGATTTGGGGGAGATCTGGTTGTGTGTGTGTGCGCTCCGAACAACACGAG
GTTGGGGAAAGAGGGTGTGGAGGGGGTGTCTATTTATTACGGCGGGCGAGGAAG
GGAAAGCGAAGGAGCGGTGGGAAAGGAATCCCCCGTAGCTGCCGGTGCCGTGA
GAGGAGGAGGAGGCCGCCTGCCGTGCCGGCTCACGTCTGCCGCTCCGCCACGCA
ATTTCTGGATGCCGACAGCGGAGCAAGTCCAACGGTGGAGCGGAACTCTCGAGA
GGGGTCCAGAGGCAGCGACAGAGATGCCGTGCCGTCTGCTTCGCTTGGCCCGAC
GCGACGCTGCTGGTTCGCTGGTTGGTGTCCGTTAGACTCGTCGATCGACGGCGTT
TAACAGGCTGGCATTATCTACTCGAAACAAGAAAAATGTTTCCTTAGTTTTTTTA
ATTTCTTAAAGGGTATTTGTTTAATTTTTAGTCACTTTATTTTATTCTATTTTATAT
CTAAATTATTAAATAAAAAAACTAAAATAGAGTTTTAGTTTTCTTAATTTAGAGG
CTAAAATAGAATAAAATAGATGTACTAAAAAAATTAGTCTATAAAAACCATTAA
CCCTAAACCCTAAATGGATGTACTAATAAAATGGATGAAGTATTATATAGGTGA
AGCTATTTGCAAAAAAAAAGGAGAACACATGCACACTAAAAAGATAAAACTGTA
GAGTCCTGTTGTCAAAATACTCAATTGTCCTTTAGACCATGTCTAACTGTTCATTT
ATATGATTCTCTAAAACACTGATATTATTGTAGTACTATAGATTATATTATTCGTA
GAGTAAAGTTTAAATATATGTATAAAGATAGATAAACTGCACTTCAAACAAGTG
TGACAAAAAAAATATGTGGTAATTTTTTATAACTTAGACATGCAATGCTCATTAT
CTCTAGAGAGGGGCACGACCGGGTCACGCTGCACTGCAG
Primer Forward-SacI
SEQ ID NO: 14
GGGGAGCTCACCTGCAGAAGTAACACCAAACAACAG
Primer Reverse-KpnI
SEQ ID NO: 15
GGGAAGCTTCTGCAGTGCAGCGTGACCCGGTCGTG
Primer Forward-HindIII
SEQ ID NO: 16
GGGAAGCTTCCTGCAGAAGTAACACCAAACAACAG
```

What is claimed is:

1. A method of increasing stress tolerance in a plant, the method comprising expressing a heterologous MG synthase gene in the plant, thereby increasing the tolerance of the plant to stress.

2. The method of claim 1, wherein the MG synthase gene encodes a protein that has at least 90% identity to the amino acid sequence SEQ ID NO:1, SEQ ID NO:2, SEQ ID NO:3, or SEQ ID NO:4.

3. The method of claim 1, wherein the MG synthase gene encodes a protein comprising the amino acid sequence of SEQ ID NO:1, SEQ ID NO:2, SEQ ID NO:3, or SEQ ID NO:4.

4. The method of claim 1, wherein the plant is corn, switchgrass, sorghum, miscanthus, sugarcane, poplar, pine, wheat, rice, soy, cotton, barley, turf grass, tobacco, potato, bamboo, rape, sugar beet, sunflower, willow, or eucalyptus.

5. The method of claim 1, wherein the MG synthase gene is operably linked to a drought-inducible promoter.

6. A plant comprising an expression cassette comprising a heterologous nucleic acid sequence encoding a polypeptide having at least 90% identity to the amino acid SEQ ID NO:1, SEQ ID NO:2, SEQ ID NO:3, or SEQ ID NO:4.

7. The plant of claim 6, wherein the heterologous nucleic acid encodes a polypeptide that comprises the amino acid sequence of SEQ ID NO:1, SEQ ID NO:2, SEQ ID NO:3, or SEQ ID NO:4.

8. The plant of claim 6, wherein the plant is corn, switchgrass, sorghum, miscanthus, sugarcane, poplar, pine, wheat, rice, soy, cotton, barley, turf grass, tobacco, potato, bamboo, rape, sugar beet, sunflower, willow, or eucalyptus.

9. The plant of claim 6, wherein the heterologous nucleic acid is operably linked to a drought-inducible promoter.

10. A method of increasing stress tolerance in a plant, the method comprising expressing a heterologous mannosyl-phospho-glycerate synthase gene in the plant, wherein the plant comprises a phosphatase that converts mannosyl-3-phophoglycerate to mannosylglycerate.

11. The method of claim 10, wherein the phosphatase is expressed by a heterologous nucleic acid present in the plant.

12. The method of claim 10, wherein the plant is corn, switchgrass, sorghum, miscanthus, sugarcane, poplar, pine, wheat, rice, soy, cotton, barley, turf grass, tobacco, potato, bamboo, rape, sugar beet, sunflower, willow, or eucalyptus.

13. A plant comprising a heterologous mannosyl-phospho-glycerate synthase gene and wherein the plant comprises a phosphatase that converts mannosyl-3-phophoglycerate to mannosylglycerate.

14. The plant of claim 13, wherein the phosphatase is encoded by a heterologous gene.


SEQUENCE LISTING (34 sequences)

SEQ ID NO: 1 - protein, 466 aa, Selaginella moellendorffii
MDDSSPPNRVLARYCQILVRAGQISGGSPREILAPASLWTILGSMSLVCFPFKEEDVAVVVRNVECAAAHPRVSTVLCVGYSKGETWCAIEAKRPSIESSTGKRIILLQQKRIGVSLRSGKGDGMNTALAYFLDHTELKRIHFYDSDIVSFSADWITKAERQADLDFDVVRHYFPRSSTDAMITWFVTKIGFCLLWPKTVLPFIEQPLGGELLLTRKAAEALYTDHRVRGQSDWGIDTLYTFIMVQKGLHLAEVYIPEGKVHALYSGLRDLRTMLVECFSAMQSLKDEAVPLNEGTHRMEYTRPVPELVKQKVGYDVEKTLKLLRSNWTQGQRDLLQKHFDPALAKGLLNASEWPTWGFADEEAWVAAYRTLLVHFEKGDEDWEELLFKIWVSRVLNHTMRHSLRGYDAALDALRSLIWETQHQSAMLSKSAAANHHIVSGHSASEALPRTAGQLREKRMDAACAV

SEQ ID NO: 2 - protein, 429 aa, Physcomitrella patens
MSLVCFPFKEEDVAVVVGNIECAASHPRVAAILCVGYSQGETWHAIEAAKPRIERATGKEIFLVLQRRIGVSLRGGKGDGMNTALAFFLEKDVYPRLHFYDADIVSFSGEWISKAEKQADLDYDIVRHYFPRSSTDAMITWLVTKLGFAMLWPNSTLPFIEQPLGGELLLTKKAAEVLYGDHRVRSQSDWGIDTLYTFVTAQAGLRLAEVYISEGKVHALYGGLRDLRTMLVECFSAVQSLRKEILPSEDGAVHCIEPAKAVPDLIKQKVGYDIEKTLKLLKSNWTPRQQELLHQHFDSALVKGLLNSAEWPCWSFADEDAWTSAYLKFLDHFEKGDSDWEELLFKVWVARVLNHTFKNVMRGYDSALGALRDLVWDTRHQFAVKLKANSVPNHAIVSGHSAAEGLVTRAQSGRKKPKFEVENLCTVQQ

SEQ ID NO: 3 - protein, 397 aa, Rhodothermus marinus
MSLVVFPFKHEHPEVLLHNVRVAAAHPRVHEVLCIGYERDQTYEAVERAAPEISRATGTPVSVRLQERLGTLRPGKGDGMNTALRYFLEETQWERIHFYDADITSFGPDWITKAEEAADFGYGLVRHYFPRASTDAMITWMITRTGFALLWPHTELSWIEQPLGGELLMRREVAAMLYEDERVRRRSDWGIDTLYTFVTVQQGVSIYECYIPEGKAHRLYGGLDDLRTMLVECFAAIQSLQHEVVGQPAIHRQEHPHRVPVHIAERVGYDVEATLHRLMQHWTPRQVELLELFTTPVREGLRTCQRRPAFNFMDEMAWAATYHVLLEHFQPGDPDWEELLFKLWTTRVLNYTMTVALRGYDYAQQYLYRMLGRYRYQAALENGRGHPVPPRAALSTA

SEQ ID NO: 4 - protein, 693 aa, Dehalococcoides ethenogenes
MRIESLRPAEHLGNVTIHGVRRFLELDSGAPSLPHLQESREVEMVDQQAISDVEKKMAIILPIKDEDLKVFEGVLSGIPHDCLMIVISNSSKQEVDNFKNEKDIVSRFCRITHRQAIVVHQKNPELAGAIADAGYPELLGKDGLIRSGKAEGMILGIILTMFSGREYVGFIDTDNYIPGAVWEYAKHFATGFNLAQSPYSMVRVLWKYKPKLVGDLYFKRWGRVSEVTNKHLNHLISSKGKFETEIIKTGNAGEHAMSIELAKRLTYGTGYAVETKEIMSILEQFSGILPIEDREVAEEGVEILQTETINPHLHEDKGGDHLIQDMLLPSLAVIYHSPLADESVKKKIENQLAGIEGLENNPEIPQVKLIPPPQKMDLAKFSAVIEKYLPQMVLPDGELISRIARPSRLPSSGQFKKVIYTDLDGTLLSPLTYSYSTALDALRLLKDKELPLVFCSAKTMGEQDLYRNELGIKDPFITENGGAIFIPKDYFRLPFAYDRVAGNYLVIELGMSYKDIRHILKKALAEACTEIENSERAGNIFITSFGDMSVEDVSRLTDLNLKQAELAKQREYSETVHIEGDKRSTNIVLNHIQQNGLEYSFGGRFYEVTGGNDKGKAIKVLNELFRLNFGNIHTFGLGDSENDYSMLETVDSPILVQRPGNKWHKMRLRNPSYVRGVGPEGFSRAVTDIILPM

SEQ ID NO: 5 - protein, 1269 aa, Selaginella moellendorfii
ATGTCGCTGGTGTGCTTTCCATTCAAGGAGGAGGACGTTGCCGTGGTCGTCCGCAACGTAGAGTGCGCCGCTGCTCACCCGCGAGTCTCCACCGTGCTGTGCGTCGGCTACAGCAAAGGCGAGACGTGGTGCGCCATCGAGGCCAAGAGACCGTCCATCGAGAGCTCCACCGGCAAGCGCATCATCCTCCTCCAGCAGAAGCGCATCGGGGTGTCGCTGCGATCCGGCAAAGGCGATGGAATGAACACGGCGCTGGCCTACTTTCTCGACCACACAGAGCTCAAGCGCATCCATTTCTACGACTCCGACATCGTCTCCTTCTCCGCGGACTGGATCACCAAGGCCGAGCGGCAGGCGGATCTAGACTTCGACGTGGTCCGGCACTACTTCCCCAGATCCAGCACCGACGCTATGATCACTTGGTTTGTAACGAAGATCGGTTTTTGCCTCCTCTGGCCCAAGACGGTGCTGCCATTCATCGAGCAGCCACTGGGCGGAGAGCTCCTACTCACGAGAAAGGCCGCCGAGGCGCTCTACACCGATCACCGAGTTCGCGGCCAGAGTGACTGGGGCATCGACACTCTCTACACTTTCATCATGGTCCAGAAGGGTCTCCACTTGGCCGAGGTCTACATCCCGGAGGGCAAGGTCCACGCCCTCTACTCGGGCCTTCGGGATCTAAGGACCATGCTGGTGGAGTGCTTCTCGGCAATGCAGTCGCTCAAGGACGAGGCGGTGCCGCTCAACGAGGGGACTCACAGGATGGAGTACACCCGGCCGGTGCCCGAGCTTGTGAAGCAAAAGGTTGGCTACGACGTGGAGAAGACGCTCAAGCTCTTGCGGAGCAACTGGACGCAGGGGCAGCGGGATCTCCTCCAGAAGCACTTCGATCCGGCGCTCGCCAAGGGGCTCCTGAATGCCAGCGAGTGGCCGACGTGGGGCTTTGCCGACGAGGAGGCGTGGGTGGCGGCGTACCGGACGCTCTTGGTTCACTTCGAGAAAGGTGACGAGGACTGGGAGGAGCTGCTGTTTAAGATCTGGGTGAGCAGGGTACTCAACCACACCATGAGGCACTCGCTCCGGGGATACGATGCTGCTCTGGATGCTCTGAGGAGCTTGATCTGGGAGACGCAGCACCAGTCTGCGATGCTGTCCAAGAGCGCTGCTGCGAATCATCACATCGTCTCGGGGCATTCAGCGTCGGAGGCTCTGCCGAGAACCGCTGGTCAGCTTCGGGAGAAACGCATGGACGCTGCCTGTGCTGTTTGA

SEQ ID NO: 6 - protein, 1290 aa, Physcomitrella patens
ATGTCTTTGGTGTGCTTCCCATTCAAGGAAGAAGATGTCGCAGTGGTGGTTGGTAATATAGAGTGCGCAGCATCGCATCCCCGGGTTGCAGCCATTTTGTGTGTGGGCTACTCGCAAGGAGAGACGTGGCACGCCATTGAAGCTGCTAAGCCACGCATTGAGAGAGCTACAGGAAAGGAAATTTTTCTGGTGCTGCAGCGACGGATTGGTGTGAGCCTTCGTGGAGGCAAAGGTGATGGCATGAACACGGCACTAGCTTTCTTTCTGGAGAAGGACGTGTATCCGCGTCTGCACTTCTACGACGCTGATATTGTCTCGTTCTCCGGAGAGTGGATTTCCAAAGCAGAGAAGCAGGCGGATCTCGACTACGATATCGTCCGTCATTACTTCCCGAGATCCAGCACGGATGCCATGATCACATGGCTTGTGACGAAGCTGGGGTTTGCTATGCTGTGGCCCAACTCGACTTTGCCTTTCATTGAGCAACCCCTCGGTGGAGAACTCCTGCTAACGAAAAAGGCGGCTGAAGTGCTCTACGGAGACCACCGTGTTCGATCTCAAAGTGACTGGGGGATAGATACTCTTTACACTTTTGTCACCGCACAAGCAGGTCTACGTCTCGCTGAGGTTTACATCTCTGAAGGGAAGGTCCATGCGCTATATGGTGGTCTTCGAGATCTCCGGACTATGCTCGTGGAATGTTTCTCAGCGGTGCAGTCGCTACGTAAGGAAATTCTTCCCAGTGAAGATGGAGCAGTTCACTGCATTGAACCAGCTAAGGCCGTCCCGGACTTGATCAAACAAAAAGTCGGGTACGACATAGAAAAGACATTGAAGCTGTTGAAGAGCAATTGGACACCTCGACAGCAGGAGCTTCTGCACCAGCATTTTGACTCTGCCCTTGTGAAGGGATTGTTGAACTCTGCCGAGTGGCCGTGCTGGAGTTTCGCAGATGAAGATGCCTGGACAAGCGCATATCTAAAATTCCTCGACCACTTCGAGAAGGGAGATAGTGATTGGGAGGAGCTTCTGTTTAAGGTTTGGGTGGCCCGGGTACTGAACCACACTTTCAAGAATGTAATGCGTGGGTACGACAGTGCCCTAGGTGCTTTGCGTGACTTGGTGTGGGACACTCGCCACCAGTTTGCTGTTAAGCTTAAAGCCAATTCCGTCCCCAACCACGCCATCGTGTCGGGCCACTCAGCTGCAGAGGGGCTGGTCACCCGAGCTCAATCCGGCCGCAAGAAACCCAAATTCGAGGTTGAAAATCTGTGTACAGTGCAACAGTAA

SEQ ID NO: 7 - protein, 1269 aa, Selaginella moellendorffii
ATGTCTCTTGTTTGTTTTCCATTCAAGGAAGAGGATGTTGCTGTTGTGGTTAGAAATGTGGAGTGCGCTGCAGCTCATCCTAGAGTGTCTACTGTTTTGTGTGTGGGTTATTCAAAGGGAGAAACATGGTGCGCTATTGAGGCAAAAAGACCATCTATCGAATCTTCAACTGGAAAGAGAATTATTCTTTTGCAACAGAAGAGAATCGGAGTTTCTCTTAGATCAGGAAAGGGAGATGGTATGAATACTGCTCTTGCATATTTTCTTGATCATACAGAGTTGAAGAGAATCCATTTCTACGATTCTGATATCGTTTCTTTCTCAGCTGATTGGATCACAAAGGCTGAAAGACAAGCAGATCTTGATTTTGATGTGGTTAGACATTACTTCCCTAGATCTTCAACTGATGCTATGATTACTTGGTTTGTTACAAAGATCGGATTCTGTCTCCTTTGGCCTAAAACAGTGCTCCCATTCATTGAACAGCCTCTTGGAGGTGAGTTGCTCCTTACTAGAAAAGCAGCTGAGGCTCTCTATACAGATCACAGAGTTAGAGGTCAATCTGATTGGGGAATCGATACTTTGTACACTTTTATTATGGTGCAGAAGGGTCTCCATCTTGCTGAAGTTTATATCCCAGAGGGTAAAGTGCACGCATTGTACTCAGGATTGAGAGATCTCAGAACTATGCTCGTTGAATGCTTCTCTGCTATGCAATCATTGAAAGATGAAGCAGTGCCTCTCAATGAGGGAACTCATAGGATGGAATATACAAGACCTGTTCCAGAGCTTGTGAAGCAAAAAGTTGGTTACGATGTGGAAAAGACTTTGAAATTGCTCAGATCTAACTGGACACAAGGACAGAGAGATCTTTTGCAGAAGCATTTTGATCCAGCTCTTGCAAAAGGTCTCCTTAACGCTTCAGAGTGGCCTACTTGGGGATTCGCTGATGAAGAGGCATGGGTTGCAGCTTATAGAACATTGCTCGTGCACTTTGAAAAGGGAGATGAGGATTGGGAAGAGCTTTTGTTCAAAATTTGGGTTTCTAGAGTGCTCAATCATACTATGAGACACTCACTTAGAGGATACGATGCAGCTCTTGATGCTTTGAGATCTCTCATTTGGGAAACACAACATCAGTCAGCTATGTTGTCTAAGTCAGCAGCTGCAAACCATCACATCGTTTCTGGTCACTCTGCTTCAGAGGCACTTCCTAGAACAGCTGGACAGTTGAGAGAAAAAAGGATGGATGCTGCATGTGCTGTTTGA

SEQ ID NO: 8 - DNA, 35 bp, Artificial Sequence
  synthetic Primer, Reverse-BstEII
gggggtnacctcaaacagcacatgcagcatccatc

SEQ ID NO: 9 - DNA, 449 bp, Unknown
  Hsp81 promoter
gatatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgtttttccctctcttcttcataaatcaacaaaacaatcacaaatctctcgaaacgctctcgaagttccaaattttctcttagcattctctttcgtttctcgtttgcgttgaatcaaagttcgttgcgat

SEQ ID NO: 10 - DNA, 34 bp, Artificial Sequence
  synthetic Primer Forward-SacI
ggggagctcgatatcggtttgaagatggcaagtg

SEQ ID NO: 11 - DNA, 34 bp, Artificial Sequence
  synthetic Primer Reverse-KpnI
gggggtaccatcgcaacgaactttgattcaacgc

SEQ ID NO: 12 - DNA, 34 bp, Artificial Sequence
  synthetic Primer Forward-HindIII
gggaagcttgatatcggtttgaagatggcaagtg

SEQ ID NO: 13 - DNA, 1509 bp, Unknown
  Ubi promoter
acctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgatcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcag

SEQ ID NO: 14 - DNA, 36 bp, Artificial Sequence
  synthetic Primer Forward-SacI
ggggagctcacctgcagaagtaacaccaaacaacag

SEQ ID NO: 15 - DNA, 35 bp, Artificial Sequence
  synthetic Primer Reverse-KpnI
gggaagcttctgcagtgcagcgtgacccggtcgtg

SEQ ID NO: 16 - DNA, 35 bp, Artificial Sequence
  synthetic Primer Forward-HindIII
gggaagcttcctgcagaagtaacaccaaacaacag

SEQ ID NO: 17 - DNA, 20 bp, Artificial Sequence
  Synthetic primer Actin-F
ctcccgctatgtatgtcgcc

SEQ ID NO: 18 - DNA, 22 bp, Artificial Sequence
  Synthetic primer Actin-R
cagaatccagcacaataccggt

SEQ ID NO: 19 - DNA, 27 bp, Artificial Sequence
  Synthetic primer Ubiqutin-F
ggccttgtataatccctgatgaataag

SEQ ID NO: 20 - DNA, 28 bp, Artificial Sequence
  Synthetic primer Ubiqutin-R
aaagagataacaggaacggaaacatagt

SEQ ID NO: 21 - DNA, 21 bp, Artificial Sequence
  Synthetic primer GT78-F
cacctctcttgtttgttttcc

SEQ ID NO: 22 - DNA, 16 bp, Artificial Sequence
  Synthetic primer GT78-R
tccagttgaagattcg

SEQ ID NO: 23 - protein, 16 aa, Artificial Sequence
  synthetic conserved region of the MG synthase polypeptide sequence
RHYFPRSSTDAMITWF

SEQ ID NO: 24 - protein, 17 aa, Artificial Sequence
  synthetic conserved region of the MG synthase polypeptide sequence
VRHYFPRXSTDAMITWF

SEQ ID NO: 25 - protein, 34 aa, Artificial Sequence
  synthetic conserved region of the MG synthase polypeptide sequence
EVYIPEGKVHALYSGLRDLRTMLVECFSAMQSLK

SEQ ID NO: 26 - protein, 33 aa, Artificial Sequence
  synthetic conserved region of the MG synthase polypeptide sequence
EXYIXEGKXHXLYXGLXDLRTMLVECFXAXQSL

SEQ ID NO: 27 - DNA, 33 bp, Artificial Sequence
  synthetic Primer Reverse-KpnI
ggggtaccatcgcaacgaactttgattcaacgc

SEQ ID NO: 28 - DNA, 23 bp, Artificial Sequence
  synthetic primer pCAMBIA2301-F
ctagagtcgacctgcaggcatgc

SEQ ID NO: 29 - DNA, 21 bp, Artificial Sequence
  synthetic primer pCAMBIA2301-R
cgatcggggaaattcgagctg

SEQ ID NO: 30 - DNA, 21 bp, Artificial Sequence
  synthetic forward primer
caccatgtctcttgtttgttt

SEQ ID NO: 31 - DNA, 21 bp, Artificial Sequence
  synthetic forward primer
cacctctcttgtttgttttcc

SEQ ID NO: 32 - DNA, 17 bp, Artificial Sequence
  synthetic reverse primer
tcaaacagcacatgcag

SEQ ID NO: 33 - protein, 128 aa, Griffithsia japonica
MSIVCFPFKKEHVPTVLRNVEIAANHPAVSLVLLAGACANDCFSEVAKAVSIKDTSSSPYPVPVRTEVQERLGTKLRAGKGDGMNSAMRYFLTAHERPENRMSAPAERLHFYDADIESFDADWITKAE

SEQ ID NO: 34 - protein, 119 aa, Griffithsia japonica
QAEYMFMDEDAWVAAYKVFLDHFVLDDEDWKELLFKAWVARVLNYTMRHVLRGYSVALQANTDMVMKLHMKRSMEVFDHHRLSSADLSSEVNSQSDAPPSPEDLEFANRHSPAVPAAVL